(19) United States
(12) Patent Application Publication
(10) Pub. No.: US 2017/0356923 A1
(43) Pub. Date: Dec. 14, 2017

(54) METHODS OF MONITORING ADHERENCE TO QUETIAPINE THERAPY

(71) Applicant: Ameritox, LLC, Baltimore, MD (US)

(72) Inventors: Gregory L. McIntire, Greensboro, NC (US); Ayodele Morris, Midland, TX (US); Erin Strickland, Greensboro, NC (US)

(21) Appl. No.: 15/581,258

(22) Filed: Apr. 28, 2017

Related U.S. Application Data
(63) Continuation of application No. 14/823,734, filed on Aug. 11, 2015, now abandoned.
(60) Provisional application No. 62/035,687, filed on Aug. 11, 2014.

(51) Int. Cl. G01N 33/94

(52) U.S. Cl. CPC G01N 33/94; G01N 2333/924; G01N 2800/52

(57) ABSTRACT

The present disclosure provides methods for monitoring subject (e.g., patient) adherence to quetiapine therapy, for example as a component of treating a subject for a mental health disorder such as schizophrenia, bipolar disorder or major depressive disorder.

### PRIORITY CLAIM

This application is a continuation of U.S. patent application Ser. No. 14/823,734 filed Aug. 11, 2015, which claims priority to U.S. Provisional Patent Application Ser. No. 62/035,687, filed Aug. 11, 2014, the entire contents of which is incorporated herein by reference and relied upon.

### TECHNICAL FIELD

The present disclosure provides methods for monitoring subject (e.g., patient) adherence to Seroquel® (quetiapine) therapy, for example as a component of treating a subject for a mental health disorder such as schizophrenia, bipolar disorder, and major depressive disorder.

### BACKGROUND

Quetiapine (Seroquel®) is an atypical antipsychotic prescribed for the treatment of acute symptoms of schizophrenia and bipolar disorder. Along with an antidepressant, is is also used to treat major depressive disorder. Nonadherence to antipsychotic medication and substance misuse have recently been reported to be more prevalent among patients with major depressive disorder or bipolar disease than those with schizophrenia. (Millet, et al., American Society of Clinical Pyschopharmacology, poster 58, June 2015). Urine drug testing has been employed by behavioral health clinicians to monitor patient compliance through analysis of drugs and their major metabolites. Typically, adherence to quetiapine therapy is monitored by evaluating levels of quetiapine and one of its plasma metabolites, 7-hydroxy quetiapine (Baselt, Disposition of Toxic Drugs and Chemicals in Man, 10^{th} ed., pp. 1754-1756 (2014)) (see Table 1 for structure). However, these molecules are present in only low levels after dosing, thus false negative monitoring results can be observed. A recent publication demonstrated the number of positives (70.5%) and negatives (29.5%) observed from a population of quetiapine patients prescribed daily dosing of Seroquel® (DeGeorge 2015). Such false negative results can improperly induce a clinician (e.g., a physician or psychiatrist) to alter a compliant subject's quetiapine therapeutic regimen when no alteration is warranted. Improved methods for assessing and monitoring a subject's adherence to quetiapine therapy are needed.

### SUMMARY

The present disclosure provides methods for monitoring patient adherence to quetiapine therapy, for example as a component of treating a subject for a mental health disorder such as schizophrenia, bipolar disorder, or major depressive disorder.

In certain embodiments, the present disclosure provides a method for monitoring quetiapine therapy in a subject, the method comprising identifying a subject who has been prescribed quetiapine therapy; analyzing a fluid sample of the subject for the presence of a quetiapine metabolite; and identifying the subject as adherent to the prescribed quetiapine therapy if the fluid sample contains the quetiapine metabolite in an amount greater than a threshold level, or as non-adherent if the fluid sample contains no quetiapine metabolite or an amount of the quetiapine metabolite below the threshold level.

In some embodiments, the present disclosure provides a method for monitoring quetiapine therapy in a subject, the method comprising identifying a subject who has been prescribed quetiapine therapy; hydrolyzing a fluid sample of the subject; analyzing the hydrolyzed fluid sample for the presence of at least one quetiapine metabolite selected from the group consisting of: quetiapine, quetiapine sulfoxide, 7-hyroxyquetiapine, and quetiapine carboxylic acid; and identifying the subject as adherent to the prescribed quetiapine therapy if the hydrolyzed fluid sample contains the quetiapine metabolite in an amount greater than a threshold level.

In one embodiment, the present disclosure provides a method for monitoring quetiapine therapy in a subject comprising of identifying a subject who has been prescribed quetiapine therapy, obtaining a fluid sample from the subject, analyzing the fluid sample for the presence of quetiapine sulfoxide, and identifying the subject as adherent to the prescribed quetiapine therapy if the fluid sample contains quetiapine sulfoxide above a threshold level or non-adherent if the fluid sample contains no quetiapine sulfoxide or an amount of quetiapine sulfoxide below a threshold level.

In another embodiment, the present disclosure provides a method for monitoring quetiapine therapy in a subject comprising of identifying a subject who has been prescribed quetiapine therapy, obtaining a fluid sample from the subject, analyzing the fluid sample for the presence of quetiapine carboxylic acid, and identifying the subject as adherent to the prescribed quetiapine therapy if the fluid sample contains quetiapine carboxylic acid above a threshold level but non-adherent if the fluid sample contains no quetiapine carboxylic acid or an amount of quetiapine carboxylic acid below a threshold level.

In yet another embodiment, the present disclosure provides a method for monitoring quetiapine therapy in a subject comprising identifying a subject who has been prescribed quetiapine therapy, obtaining a fluid sample from the subject, analyzing the fluid sample for the presence of quetiapine sulfoxide and quetiapine carboxylic acid, and identifying the subject as adherent to the prescribed quetiapine therapy if the fluid sample contains a sum of quetiapine sulfoxide and quetiapine carboxylic acid above a threshold level but non-adherent if the fluid sample contains no quetiapine sulfoxide or quetiapine carboxylic acid or a sum of quetiapine sulfoxide and quetiapine carboxylic acid below a threshold level.

In yet another embodiment, the present disclosure provides a method for monitoring quetiapine therapy in a subject comprising of identifying a subject who has been prescribed quetiapine therapy, obtaining a fluid sample from the subject, analyzing the fluid sample for the presence of quetiapine, 7-hydroxy quetiapine, quetiapine sulfoxide, and quetiapine carboxylic acid, and identifying the subject as adherent to the prescribed quetiapine therapy if the fluid sample contains some, if not all, of the metabolites (i.e., parent drug=metabolite number 1) above a threshold level. Or, they can be determined to be adherent if the sum of quetiapine, 7-hydroxy quetiapine, quetiapine sulfoxide, and quetiapine carboxylic acid is above a threshold level but non-adherent if the fluid sample exhibits a sum of quetiapine sulfoxide and quetiapine carboxylic acid below a threshold level.

In yet another embodiment, the present disclosure provides a method for monitoring quetiapine therapy in a subject comprising of identifying a subject who has been prescribed quetiapine therapy, obtaining a fluid sample from the subject, analyzing the fluid sample for the presence of quetiapine, 7-hydroxy quetiapine, quetiapine sulfoxide, and quetiapine carboxylic acid, and identifying the subject as adherent to the prescribed quetiapine therapy if the fluid sample contains some, if not all, of the metabolites (i.e., parent drug=metabolite number 1) above a threshold level. As shown below in Table 3, lower doses of quetiapine more readily demonstrate observable levels of quetiapine sulfoxide and quetiapine carboxylic acid. As the dosage increases levels of all metabolites are observed in patient samples, as would be expected. It can also be observed, as shown in Table 21, that upon hydrolysis, even at low doses, the quetiapine and 7-hydroxy quetiapine are more readily observed along with the quetiapine sulfoxide and quetiapine carboxylic acid. However, the quetiapine sulfoxide and quetiapine carboxylic acid appear in greater abundance than the quetiapine and 7-hydroxy quetiapine. Thus, the use of quetiapine sulfoxide and quetiapine carboxylic acid to determine adherence is probably more important at low doses where quetiapine and 7-hydroxy quetiapine are difficult to observe without hydrolysis.

In another embodiment, the present disclosure provides a method of evaluating compliance with quetiapine therapy in a subject, the method comprising of obtaining a fluid sample (e.g., urine) from the subject, analyzing the fluid sample for presence or absence of an analyte, and identifying the subject as compliant if the analyte is present in the fluid sample above a threshold level The threshold level can be determined as a function of the analytical method used to assay the analyte or it can be a clinically relevant level below which the data have little clinical meaning.

In any of the methods herein, a non-compliant subject can further be counseled as to the importance of compliance and strategies for achieving compliance.

### DETAILED DESCRIPTION

While the present invention is capable of being embodied in various forms, the description below of several embodiments is made with the understanding that the present disclosure is to be considered as an exemplification of the invention, and is not intended to limit the invention to the specific embodiments illustrated. Headings are provided for convenience only and are not to be construed to limit the invention in any manner. Embodiments illustrated under any heading may be combined with embodiments illustrated under any other heading.

The use of numerical values in the various quantitative values specified in this application, unless expressly indicated otherwise, are stated as approximations as though the minimum and maximum values within the stated ranges were both preceded by the word “about.” Also, the disclosure of ranges is intended as a continuous range including every value between the minimum and maximum values recited as well as any ranges that can be formed by such values. Also disclosed herein are any and all ratios (and ranges of any such ratios) that can be formed by dividing a disclosed numeric value into any other disclosed numeric value. Accordingly, the skilled person will appreciate that many such ratios, ranges, and ranges of ratios can be unambiguously derived from the numerical values presented herein and in all instances such ratios, ranges, and ranges of ratios represent various embodiments of the present invention.

Quetiapine (Seroquel®) is an atypical antipsychotic prescribed for the treatment of acute symptoms of schizophrenia, bipolar disorder, and major depressive disorder. Quetiapine (2-(2-(4-dibenzo[b,f][1,4]thiazepine-11-yl-1-piperazinyl)ethoxy) ethanol) has a molecular weight of 383.5099 g/mol, and empirical formula of C₂₁H₂₅N₃O₂S, a calculated log P of 1.59 at pH 5.5, a CAS number of 111974-69-7, a mass-to-charge ratio (m/z) of 384.5 when ionized with the addition of a proton (ESI MS), and has a structure shown below:

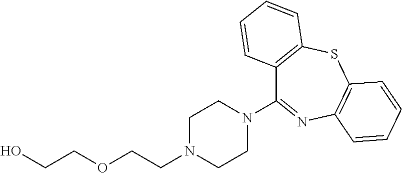 [SMILES: OCCOCCN1CCN(C2=Nc3ccccc3Sc3ccccc32)CC1]

Quetiapine is commercially available as 25 mg, 50 mg, 100 mg, 200 mg, and 400 mg tablets. It is rapidly absorbed after oral administration. Dosing is recommended to be either without food or with a light meal (˜300 calories) as this can increase the bioavailability by ˜20%. The mean elimination half-life is 6 hours. Steady state serum concentrations for quetiapine are typically achieved after 2 days of dosing.

Quetiapine is metabolized in the liver primarily by CYP3A4. Metabolism includes oxidative N-dealkylation, hydroxylation of the 7 position on the ring, S-oxidation, and oxidation of the alkyl OH group to the corresponding carboxylic acid. Nearly twenty metabolites of quetiapine have been previously identified including those conjugated to glucuronic acid. Select metabolites of quetiapine are shown in Table 1 below.

TABLE 1
Quetiapine and Select Metabolites Thereof.

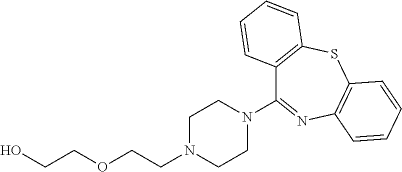 [SMILES: OCCOCCN1CCN(C2=Nc3ccccc3Sc3ccccc32)CC1]

Quetiapine

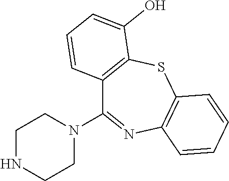 [SMILES: Oc1cccc2c1Sc1ccccc1N=C2N1CCNCC1]

7-Hydroxy N-Desalkyl Quetiapine

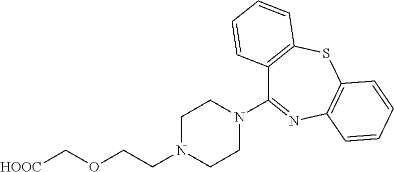 [SMILES: O=C(O)COCCN1CCN(C2=Nc3ccccc3Sc3ccccc32)CC1]

Quetiapine Carboxylic Acid

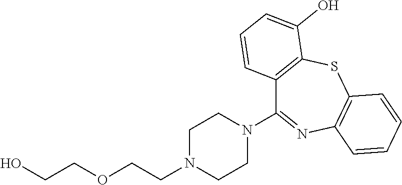 [SMILES: OCCOCCN1CCN(C2=Nc3ccccc3Sc3c(O)cccc32)CC1]

7-Hydroxy Quetiapine

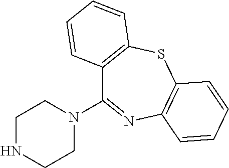 [SMILES: c1ccc2c(c1)N=C(N1CCNCC1)c1ccccc1S2]

N-Desalkyl Quetiapine
(Norquetiapine)

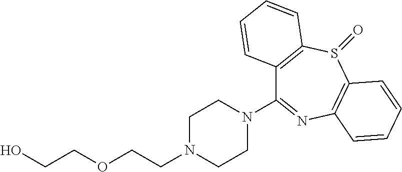 [SMILES: O=S1c2ccccc2N=C(N2CCN(CCOCCO)CC2)c2ccccc21]

Quetiapine Sulfoxide

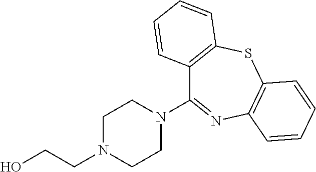 [SMILES: OCCN1CCN(C2=Nc3ccccc3Sc3ccccc32)CC1]

O-Desalkyl Quetiapine

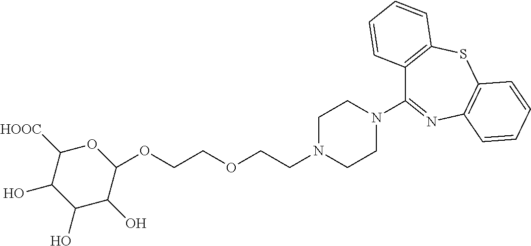 [SMILES: O=C(O)C1OC(OCCOCCN2CCN(C3=Nc4ccccc4Sc4ccccc43)CC2)C(O)C(O)C1O]

Quetiapine Glucuronide

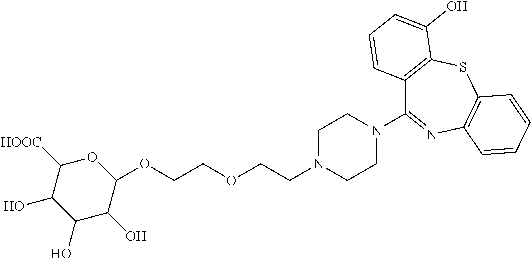 [SMILES: O=C(O)C1OC(OCCOCCN2CCN(C3=Nc4ccccc4Sc4c(O)cccc43)CC2)C(O)C(O)C1O]

7-Hydroxy Quetiapine Glucuronide

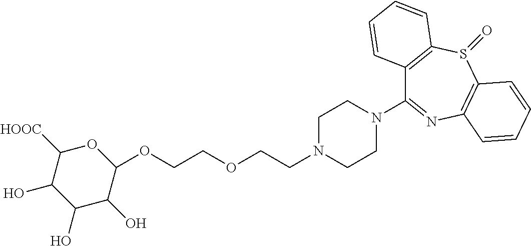 [SMILES: O=C(O)C1OC(OCCOCCN2CCN(C3=Nc4ccccc4S(=O)c4ccccc43)CC2)C(O)C(O)C1O]

Quetiapine Sulfoxide Glucuronide

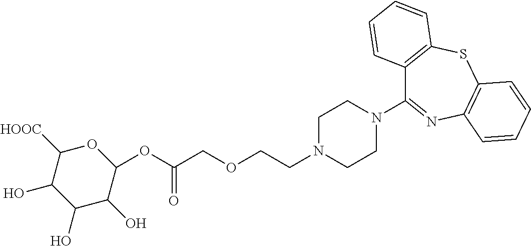 [SMILES: O=C(COCCN1CCN(C2=Nc3ccccc3Sc3ccccc32)CC1)OC1OC(C(=O)O)C(O)C(O)C1O]

Quetiapine Carboxylic Acid Glucuronide

Among the various known quetiapine metabolites, the carboxylic acid and the sulfoxide are not biologically active, but have been reported to be “major” metabolites observed in urine (e.g., defined by ≧0% total drug exposure) (Baselt 2014). Metabolites 7-hydroxy quetiapine and N-desalkyl quetiapine are known to be biologically active, but are observable in only small quantities in the urine of in humans.

Quetiapine metabolite designated carboxylic acid is the result of oxidation of the terminal alkyl OH group. Together with the sulfoxide, these metabolites have been reported to be primary metabolites in the urine (Baselt 2014). However, earlier work using GC/MS did not use them to monitor quetiapine levels inasmuch as the carboxylic acid has low volatility which is required to maintain the molecule in the gas phase for analysis by GC/MS. The sulfoxide proved to be unstable at temperatures required for GC analysis (Reference?). As such, reference standards for them were not as readily available as for 7-hydroxy quetiapine and N-desalkyl quetiapine. It is noteworthy that quetiapine and the 7-hydroxy, carboxylic acid, and sulfoxide metabolites can also exist as glucuronide conjugates in the urine (see Table 1).

Drug adherence has been shown to be particularly low in patients with schizophrenia, bipolar disorder, and major depressive disorder (Millet, et al., American Society of Clinical Pyschopharmacology, Poster 58, June 2015) Urine drug testing has been employed by behavioral health clinicians to monitor patient compliance through analysis of drugs and their major metabolites.

In one embodiment, the present disclosure provides a method for monitoring quetiapine therapy in a subject. In some embodiments, the method comprises of identifying a subject who has been prescribed quetiapine therapy, obtaining a fluid sample from the subject, analyzing the fluid sample for the presence of quetiapine carboxylic acid, and identifying the subject as adherent to the prescribed quetiapine therapy if the fluid sample contains quetiapine carboxylic acid and/or any other metabolite or parent drug above a threshold level but non-adherent if the fluid sample contains no quetiapine carboxylic acid or an amount of quetiapine carboxylic acid and/or any other metabolite or parent drug below a threshold level. In some embodiments, the method further comprises counseling the subject on dangers of non-adherence to quetiapine therapy or strategies to achieve compliance if the subject is identified as non-adherent. In some embodiments, the threshold level is a minimum detectable amount of quetiapine carboxylic acid. In some embodiments, the threshold level is about 5 ng/mL to about 500 ng/mL, for example about 5 ng/mL, about 10 ng/mL, about 15 ng/mL, about 20 ng/mL, about 25 ng/mL, about 30 ng/mL, about 35 ng/mL, about 40 ng/mL, about 45 ng/mL, about 50 ng/mL, about 55 ng/mL, about 60 ng/mL, about 65 ng/mL, about 70 ng/mL, about 75 ng/mL, about 80 ng/mL, about 85 ng/mL, about 90 ng/mL, about 95 ng/mL, about 100 ng/mL, about 125 ng/mL, about 150 ng/mL, about 175 ng/mL, about 200 ng/mL, about 225 ng/mL, about 250 ng/mL, about 275 ng/mL, about 300 ng/mL, about 325 ng/mL, about 350 ng/mL, about 375 ng/mL, about 400 ng/mL, about 425 ng/mL, about 450 ng/mL, about 475 ng/mL, or about 500 ng/mL. In some embodiments, the threshold level is about 5 ng/mL. In some embodiments, the fluid sample is a urine sample.

In another embodiment, the present disclosure provides a method for monitoring quetiapine therapy in a subject. In some embodiments, the method comprises of identifying a subject who has been prescribed quetiapine therapy, obtaining a fluid sample from the subject, analyzing the fluid sample for the presence of quetiapine sulfoxide, and identifying the subject as adherent to the prescribed quetiapine therapy if the fluid sample contains quetiapine sulfoxide above a threshold level but non-adherent if the fluid sample contains no quetiapine sulfoxide or an amount of quetiapine sulfoxide below a threshold level. In some embodiments, the method further comprises counseling the subject on dangers of non-adherence to quetiapine therapy if the subject is identified as non-adherent. In some embodiments, the threshold level is a minimum detectable amount of quetiapine sulfoxide. In some embodiments, the threshold level is about 5 ng/mL to about 500 ng/mL, for example about 5 ng/mL, about 10 ng/mL, about 15 ng/mL, about 20 ng/mL, about 25 ng/mL, about 30 ng/mL, about 35 ng/mL, about 40 ng/mL, about 45 ng/mL, about 50 ng/mL, about 55 ng/mL, about 60 ng/mL, about 65 ng/mL, about 70 ng/mL, about 75 ng/mL, about 80 ng/mL, about 85 ng/mL, about 90 ng/mL, about 95 ng/mL, about 100 ng/mL, about 125 ng/mL, about 150 ng/mL, about 175 ng/mL, about 200 ng/mL, about 225 ng/mL, about 250 ng/mL, about 275 ng/mL, about 300 ng/mL, about 325 ng/mL, about 350 ng/mL, about 375 ng/mL, about 400 ng/mL, about 425 ng/mL, about 450 ng/mL, about 475 ng/mL, or about 500 ng/mL. In some embodiments, the threshold level is about 5 ng/mL. In some embodiments, the fluid sample is a urine sample.

In another embodiment, the present disclosure provides a method for monitoring quetiapine therapy in a subject. In some embodiments, the method comprises of identifying a subject who has been prescribed quetiapine therapy, obtaining a fluid sample from the subject, analyzing the fluid sample for the presence of quetiapine sulfoxide and quetiapine carboxylic acid, and identifying the subject as adherent to the prescribed quetiapine therapy if the fluid sample contains a sum of quetiapine sulfoxide and quetiapine carboxylic acid above a threshold level but non-adherent if the fluid sample contains no quetiapine sulfoxide or quetiapine carboxylic acid or a sum of quetiapine sulfoxide and quetiapine carboxylic acid below a threshold level. In some embodiments, the method further comprises counseling the subject on dangers of non-adherence to quetiapine therapy or strategies to achieve compliance if the subject is identified as non-adherent. In some embodiments, the threshold level is a minimum detectable amount of quetiapine sulfoxide or quetiapine carboxylic acid. In some embodiments, the threshold level is about 5 ng/mL to about 500 ng/mL, for example about 5 ng/mL, about 10 ng/mL, about 15 ng/mL, about 20 ng/mL, about 25 ng/mL, about 30 ng/mL, about 35 ng/mL, about 40 ng/mL, about 45 ng/mL, about 50 ng/mL, about 55 ng/mL, about 60 ng/mL, about 65 ng/mL, about 70 ng/mL, about 75 ng/mL, about 80 ng/mL, about 85 ng/mL, about 90 ng/mL, about 95 ng/mL, about 100 ng/mL, about 125 ng/mL, about 150 ng/mL, about 175 ng/mL, about 200 ng/mL, about 225 ng/mL, about 250 ng/mL, about 275 ng/mL, about 300 ng/mL, about 325 ng/mL, about 350 ng/mL, about 375 ng/mL, about 400 ng/mL, about 425 ng/mL, about 450 ng/mL, about 475 ng/mL, or about 500 ng/mL. In some embodiments, the threshold level is about 10 ng/mL. In some embodiments, the fluid sample is a urine sample.

In another embodiment, the present disclosure provides a method for monitoring quetiapine therapy in a subject. In some embodiments, the method comprises of identifying a subject who has been prescribed quetiapine therapy, obtaining a fluid sample from the subject, analyzing the fluid sample for the presence of quetiapine, quetiapine sulfoxide, N-desalkylquetiapine, 7-hyroxyquetiapine, 7-hydroxyquetiapine glucuronide, quetiapine carboxylic acid, quetiapine glucuronide, quetiapine sulfoxide glucuronide, and quetiapine carboxylic acid glucuronide, and identifying the subject as adherent to the prescribed quetiapine therapy if the fluid sample contains a sum of quetiapine, quetiapine sulfoxide, N-desalkylquetiapine, 7-hyroxyquetiapine, 7-hydroxyquetiapine glucuronide, quetiapine carboxylic acid, quetiapine glucuronide, quetiapine sulfoxide glucuronide, and quetiapine carboxylic acid glucuronide above a threshold level but non-adherent if the fluid sample contains a sum of quetiapine sulfoxide and quetiapine carboxylic acid below a threshold level. In some embodiments, the patient can be defined as adherent if any of quetiapine, quetiapine sulfoxide, N-desalkylquetiapine, 7-hyroxyquetiapine, 7-hydroxyquetiapine glucuronide, quetiapine carboxylic acid, quetiapine glucuronide, quetiapine sulfoxide glucuronide, and quetiapine carboxylic acid glucuronide are found in the fluid sample above a threshold. In other embodiments, the patient can be defined as adherent if quetiapine sulfoxide and quetiapine carboxylic acid are found above a threshold at low doses where quetiapine and 7-hydroxy quetiapine are not found. In some embodiments, the method further comprises counseling the subject on dangers of non-adherence to quetiapine therapy or strategies to achieve compliance if the subject is identified as non-adherent. In some embodiments, the threshold level is a minimum detectable amount of quetiapine sulfoxide or quetiapine carboxylic acid. In some embodiments, the threshold level is about 5 ng/mL to about 500 ng/mL, for example about 5 ng/mL, about 10 ng/mL, about 15 ng/mL, about 20 ng/mL, about 25 ng/mL, about 30 ng/mL, about 35 ng/mL, about 40 ng/mL, about 45 ng/mL, about 50 ng/mL, about 55 ng/mL, about 60 ng/mL, about 65 ng/mL, about 70 ng/mL, about 75 ng/mL, about 80 ng/mL, about 85 ng/mL, about 90 ng/mL, about 95 ng/mL, about 100 ng/mL, about 125 ng/mL, about 150 ng/mL, about 175 ng/mL, about 200 ng/mL, about 225 ng/mL, about 250 ng/mL, about 275 ng/mL, about 300 ng/mL, about 325 ng/mL, about 350 ng/mL, about 375 ng/mL, about 400 ng/mL, about 425 ng/mL, about 450 ng/mL, about 475 ng/mL, or about 500 ng/mL. In some embodiments, the threshold level is about 5 ng/mL. In some embodiments, the fluid sample is a urine sample.

In some embodiments, the present disclosure provides a method for monitoring quetiapine therapy in a subject. In some embodiments, the method comprises identifying a subject who has been prescribed quetiapine therapy; analyzing a fluid sample of the subject for the presence of a quetiapine metabolite; and identifying the subject as adherent to the prescribed quetiapine therapy if the fluid sample contains the quetiapine metabolite in an amount greater than a threshold level, or as non-adherent if the fluid sample contains no quetiapine metabolite or an amount of the quetiapine metabolite below the threshold level. In some embodiments, the method further comprising counseling the subject on dangers of non-adherence to quetiapine therapy if the subject is identified as non-adherent. In some embodiments, the threshold level is a minimum detectable amount of the quetiapine metabolite. In some embodiments, the threshold level is about 50 ng/mL, more preferably 20 ng/mL and most preferably 5 ng/mL. In some embodiments, the fluid sample is a urine sample. In some embodiments, the quetiapine metabolite is one or more of: quetiapine, quetiapine sulfoxide, N-desalkylquetiapine, 7-hyroxyquetiapine, 7-hydroxyquetiapine glucuronide, quetiapine carboxylic acid, quetiapine glucuronide, quetiapine sulfoxide glucuronide, and quetiapine carboxylic acid glucuronide. In some embodiments, the quetiapine metabolite is two or more of: quetiapine, quetiapine sulfoxide, N-desalkylquetiapine, 7-hyroxyquetiapine, 7-hydroxyquetiapine glucuronide, quetiapine carboxylic acid, quetiapine glucuronide, quetiapine sulfoxide glucuronide, and quetiapine carboxylic acid glucuronide. In some embodiments, the quetiapine metabolite is three or more of: quetiapine, quetiapine sulfoxide, N-desalkylquetiapine, 7-hyroxyquetiapine, 7-hydroxyquetiapine glucuronide, quetiapine carboxylic acid, quetiapine glucuronide, quetiapine sulfoxide glucuronide, and quetiapine carboxylic acid glucuronide. In some embodiments, the quetiapine metabolite is four or more of: quetiapine, quetiapine sulfoxide, N-desalkylquetiapine, 7-hyroxyquetiapine, 7-hydroxyquetiapine glucuronide, quetiapine carboxylic acid, quetiapine glucuronide, quetiapine sulfoxide glucuronide, and quetiapine carboxylic acid glucuronide. In some embodiments, the quetiapine metabolite is quetiapine, quetiapine sulfoxide, 7-hyroxyquetiapine, and quetiapine carboxylic acid. In some embodiments, the subject is identified as adherent to the prescribed quetiapine therapy if the fluid sample contains quetiapine, quetiapine sulfoxide, 7-hyroxyquetiapine, and quetiapine carboxylic acid in an amount greater than or equal to the threshold value, wherein the threshold value is 5 ng/mL. In some embodiments, the method further comprises contacting the fluid sample with a hydrolyzing enzyme before analyzing the fluid sample for the presence of the quetiapine metabolite. In some embodiments, the hydrolyzing enzyme is a glucuronidase enzyme. In some embodiments, the glucuronidase enzyme is a β-glucuronidase enzyme. In some embodiments, the β-glucuronidase enzyme is a naturally occurring β-glucuronidase enzyme. In other embodiments, the β-glucuronidase enzyme is a recombinant β-glucuronidase enzyme. In some embodiments, the method further comprises generating a report including a statement identifying the subject as adherent or non-adherent. In some embodiments, the report further includes a recommendation to modify the prescribed quetiapine therapy if the subject is identified as non-adherent. In some embodiments, the report includes a recommendation to obtain a genetic test to determine a biological cause for the subject's non-adherence if the subject is identified as non-adherent. In some embodiments, the recommendation to obtain a genetic test includes a recommendation to obtain a genetic test identifying at least one (e.g., any) genetic variant in the subject's cytochrome P450 3A4 (CYP3A4) gene resulting in altered drug metabolism. Genetic testing (e.g., genotyping) for metabolic enzymes of the cytochrome P450 family can be used to determine whether a patient has the metabolic capability to handle quetiapine as well as a number of mental health and pain medications. In the exact case of quetiapine, the primary metabolic pathway is through CYP3A4. If the subject is deficient in CYP3A4 capacity, it may be better to prescribe an alternative antipsychotic. If, however, the subject is a normal metabolizer via CYP3A4, he or she may be diverting or misusing their prescription if the determined drug ratio is outside compliance. Rapid metabolizers may also have genetic issues wherein the drug is metabolized so quickly that they might not fit a “normal” standard distribution of drug ratios. Thus, genetic testing can offer rationale for why certain patients appear to be “not adherent” to their prescribed medications. Such a genetic test could therefore reveal a biological cause to quetiapine metabolite ratios that would suggest an otherwise adherent subject is non-adherent.

In other embodiments, the present disclosure provides a method for monitoring quetiapine therapy in a subject, the method comprising identifying a subject who has been prescribed quetiapine therapy; hydrolyzing a fluid sample of the subject; analyzing the hydrolyzed fluid sample for the presence of at least one quetiapine metabolite selected from the group consisting of: quetiapine, quetiapine sulfoxide, 7-hyroxyquetiapine, and quetiapine carboxylic acid; and identifying the subject as adherent to the prescribed quetiapine therapy if the hydrolyzed fluid sample contains the quetiapine metabolite in an amount greater than a threshold level. In some embodiments, the step of hydrolyzing the fluid sample comprises contacting the fluid sample with a composition comprising a hydrolyzing enzyme. In some embodiments, the hydrolyzing enzyme is a glucuronidase enzyme. In some embodiments, the glucuronidase enzyme is a β-glucuronidase enzyme. In some embodiments, the β-glucuronidase enzyme is a naturally occurring β-glucuronidase enzyme. In other embodiments, the β-glucuronidase enzyme is a recombinant β-glucuronidase enzyme. In some embodiments, the threshold value is 5 ng/mL. In some embodiments, the method further comprises identifying the subject as non-adherent if the hydrolyzed fluid sample does not contain any one of quetiapine, quetiapine sulfoxide, 7-hyroxyquetiapine, and quetiapine carboxylic acid in an amount above the threshold value. In some embodiments, the method further comprises generating a report including a statement identifying the subject as adherent or non-adherent. In some embodiments, the report further includes a recommendation to modify the prescribed quetiapine therapy if the subject is identified as non-adherent. In some embodiments, the report includes a recommendation to obtain a genetic test to determine a biological cause for the subject's non-adherence if the subject is identified as non-adherent. In some embodiments, the recommendation to obtain a genetic test includes a recommendation to obtain a genetic test identifying at least one (e.g., any) genetic variant in the subject's CYP450 enzyme 3A4.

In another embodiment, the present disclosure provides a method of evaluating compliance with quetiapine therapy in a subject. In some embodiments, the method comprises of obtaining a fluid sample from the subject, analyzing the fluid sample for presence or absence of an analyte, and identifying the subject as compliant if the analyte is present in the fluid sample. In some embodiments, the analyte comprises quetiapine and/or a quetiapine metabolite or metabolites. In some embodiments, the analyte is selected from the group consisting of quetiapine sulfoxide, quetiapine carboxylic acid, 7-hydroxy quetiapine, N-desalkyl quetiapine, or a combination thereof. In some embodiments, the analyte comprises quetiapine carboxylic acid. In some embodiments, the analyte comprises quetiapine sulfoxide. In some embodiments, the analyte comprises a combination of quetiapine carboxylic acid and quetiapine sulfoxide. In some embodiments, the analyte is considered present in the fluid sample if the analyte is detected above a threshold value. In some embodiments, the threshold value is about 50 ng/mL. In other embodiments, the threshold value is about 25 ng/mL. In still other embodiments, the threshold value is about 5 ng/mL.

### EXAMPLES

### Example 1

Urine samples of normally metabolizing human subjects who were known to be taking chronic doses of quetiapine were tested for the presence of quetiapine and 7 metabolites.

100 μL of each patient specimen was diluted with 450 μL of 250 ng/mL of hydrocodone-D6 (internal standard) in methanol and 350 μL of 0.1% formic acid. Samples were subsequently vortexed and centrifuged prior to the injection of 5 μL of each replicateon the Q-ToF. Each patient sample was analyzed twice to ensure accuracy.

In plasma, N-desalkyl quetiapine has been reported to account for 10% of the total radioactivity post oral dosing while quetiapine represents 24% of the total radioactivity, and 7-hydroxy quetiapine represents 11% of the total radioactive dose in the plasma. Excretion studies in humans report that the drug is excreted with 20% in the feces and 73% recovered in the urine.

Surprisingly, neither metabolite N-desalkyl quetiapine nor metabolite 7-hydroxy quetiapine was found to be the dominant metabolite excreted through human urine in the majority of samples. Instead, the identity of detectable quetiapine metabolites varies from subject to subject, as shown in Table 2 below:

TABLE 2
Quetiapine Metabolite Distribution in Human Urine

| | Analyte | | | | | | | |
|---|---|---|---|---|---|---|---|---|
| | 7-Hydroxy | | | | | | | |
| | Desalkyl | 7-Hydroxy | Carboxy | N-Desalkyl | O-Desalkyl | | Quetiapine | Quetiapine |
| Patient | Quetiapine | Quetiapine | Quetiapine | Quetiapine | Quetiapine | Quetiapine | Glucuronide | Sulfoxide |
| 1 | 7.27 | 0.65 | 6.71 | 8.48 | 0.00 | 0.19 | 0.40 | 1.81 |
| 2 | 23.21 | 7.76 | 43.61 | 16.66 | 0.73 | 2.08 | 6.23 | 10.81 |
| 3 | 5.22 | 0.00 | 27.86 | 11.15 | 1.08 | 8.21 | 0.02 | 2.02 |
| 4 | 10.36 | 6.86 | 26.14 | 4.18 | 0.23 | 1.87 | 5.23 | 12.90 |
| 5 | 0.39 | 0.08 | 5.36 | 0.80 | 0.00 | 0.08 | 0.52 | 0.23 |
| 6 | 5.16 | 1.23 | 34.19 | 2.94 | 0.00 | 0.58 | 3.16 | 4.10 |
| 7 | 2.25 | 0.86 | 10.95 | 4.09 | 0.04 | 0.85 | 1.06 | 2.22 |
| 8 | 7.24 | 0.54 | 51.31 | 13.44 | 0.00 | 0.31 | 4.74 | 1.79 |
| 9 | 7.95 | 3.73 | 13.07 | 8.81 | 0.30 | 0.98 | 1.86 | 6.54 |
| 10 | 5.22 | 0.35 | 3.60 | 5.20 | 0.00 | 0.12 | 0.29 | 1.09 |
| 11 | 3.21 | 0.15 | 8.23 | 2.09 | 0.00 | 0.01 | 1.18 | 0.58 |
| 12 | 0.04 | 0.04 | 0.75 | 0.08 | 0.00 | 0.06 | 0.09 | 0.12 |

*Values are analyte responses relative to an internal standard that was present in every sample. This is meant to relate relative abundance only and not meant for quantitation.

As shown above, several metabolites were present in all 12 of these random patient positive samples. This includes quetiapine, 7-hydroxyquetiapine, 7-hydroxy desalkyl quetiapine, N-desalkyl quetiapine, quetiapine sulfoxide, and carboxy quetiapine (quetiapine carboxylic acid). Quetiapine carboxylic acid and quetiapine sulfoxide were present in significant amounts in every sample.

These data demonstrate that regardless of prescribed dose, quetiapine metabolite quetiapine carboxylic acid (carboxy quetiapine) provides a greater level of sensitivity and consistency among subjects on quetiapine therapy, and therefore provides a superior urine analyte for evaluation of a subject's compliance with a quetiapine therapeutic regimen.

These data demonstrate that quetiapine metabolite quetiapine sulfoxide provides a greater level of sensitivity and consistency among subjects on quetiapine therapy, and therefore provides a superior urine analyte for evaluation of a subject's compliance with a quetiapine therapeutic regimen.

These data demonstrate that quetiapine metabolites quetiapine carboxylic acid (carboxy quetiapine) and quetiapine sulfoxide together provide a greater level of sensitivity and consistency among subjects on quetiapine therapy, and therefore provides superior urine analytes for evaluation of a subject's compliance with a quetiapine therapeutic regimen.

### Example 2

The urine of 16 patients who were prescribed 25 mg of Seroquel® (quetiapine) was tested for compliance (Table 3). 100 μL of each patient specimen was diluted with 50 μL of 1.6 μg/mL of quetiapine-D8 (internal standard) in methanol and 350 μL of 0.1% formic acid. Samples were subsequently vortexed and centrifuged prior to the injection of 5 μL on the QQQ. Each patient sample was analyzed twice to ensure accuracy.

The preponderance of quetiapine carboxylic acid and quetiapine sulfoxide in the urine as the major metabolic urine compounds is shown in Table 3. Assuming 16 opportunities to determine the patient to be either positive or negative for Seroquel® dosing, the use of quetiapine carboxylic acid and quetiapine sulfoxide resulted in 100% correct identification of those taking the prescribed medicine. The data in Table 4 demonstrate the “normal” nature of the sample validity criteria (i.e., pH, specific gravity, and creatinine). Without the quetiapine carboxylic acid and quetiapine sulfoxide metabolites, only ˜63% were determined to be positive solely by the parent compound quetiapine and ˜69% were determined to be positive when using the parent compound quetiapine in conjunction with 7-hydroxy quetiapine. Clearly, the use of all 4 analytes results in 100% correct identification of those prescribed this dose. Thus, use of quetiapine carboxylic acid and quetiapine sulfoxide as a urine biomarker at this low dose adds value to compliance monitoring for Seroquel®.

TABLE 3
Test Results from Patients Prescribed 25 mg/day Seroquel ® (all parent and
metabolite data reported in ng/mL).

| | | | | | | Quetiapine | | |
|---|---|---|---|---|---|---|---|---|
| | Specific | | Creatinine | | 7-Hydroxy | Carboxylic | Quetiapine | All 4 |
| Subject | Gravity | pH | (mg/dL) | Quetiapine | Quetiapine | Acid | Sulfoxide | mets |
| A | 1.007 | 7.9 | 45.4 | ND | ND | 94 | 22 | + |
| B | 1.022 | 5.5 | 273.7 | ND | ND | 9 | ND | + |
| C | 1.010 | 7.2 | 80.2 | 5 | ND | 493 | 202 | + |
| D | 1.009 | 8.0 | 53.8 | 40 | 18 | 492 | 343 | + |
| E | 1.012 | 6.3 | 106.9 | ND | ND | 17 | 8 | + |
| F | 1.016 | 5.8 | 242.5 | 125 | 61 | 1844 | 3738 | + |
| G | 1.009 | 7.7 | 66.5 | ND | ND | 138 | 34 | + |
| H | 1.003 | 7.0 | 34.4 | ND | 6 | 82 | 59 | + |
| I | 1.012 | 6.8 | 141.9 | 19 | 5 | 1998 | 300 | + |
| J | 1.013 | 5.2 | 152.0 | 33 | 59 | 4470 | 1411 | + |
| K | 1.004 | 7.1 | 43.4 | 19 | 7 | 2977 | 3050 | + |
| L | 1.008 | 6.4 | 59.0 | 22 | 18 | 2877 | 2679 | + |
| M | 1.013 | 7.1 | 193.9 | 196 | 38 | 5681 | 517 | + |
| N | 1.005 | 6.2 | 43.6 | ND | ND | 60 | 11 | + |
| O | 1.006 | 6.6 | 25.9 | 14 | 17 | 570 | 101 | + |
| P | 1.016 | 6.8 | 193.5 | 23 | 29 | 1308 | 1017 | + |

ND: Indicates that the listed parent or metabolite compound was either not detected or detected below the established cut-off of 5 ng/mL indicating a negative result for that particular subject.

TABLE 4
Sample Validity and Urine Results Summary at 25 mg/day (all parent and
metabolite data reported in ng/mL).

| | | | | | | Quetiapine | |
|---|---|---|---|---|---|---|---|
| | Specific | | Creatinine | | 7-Hydroxy | Carboxylic | Quetiapine |
| | Gravity | pH | (mg/dL) | Quetiapine | Quetiapine | Acid | Sulfoxide |
| Average | 1.010 | 6.7 | 109.8 | 49.8 | 25.5 | 1444.3 | 899.4 |
| Standard | 0.005 | 0.8 | 77.3 | 58.4 | 19.8 | 1695.1 | 1207.6 |
| Deviation | | | | | | | |
| Maximum | 1.022 | 8.0 | 273.7 | 196.4 | 61.2 | 5681.4 | 3738.2 |
| Value | | | | | | | |
| Median | 1.010 | 6.8 | 73.4 | 22.8 | 17.5 | 531.7 | 299.5 |
| Value | | | | | | | |
| Minimum | 1.003 | 5.2 | 25.9 | 5.3 | 5.0 | 8.7 | 8.4 |
| Value | | | | | | | |

### Example 3

The urine of 45 patients who were prescribed 50 mg of Seroquel® (quetiapine) was tested for compliance (Table 5). 100 μL of each patient specimen was diluted with 50 μL of 1.6 μg/mL of quetiapine-D8 (internal standard) in methanol and 350 μL of 0.1% formic acid. Samples were subsequently vortexed and centrifuged prior to the injection of 5 μL on the QQQ.

The preponderance of quetiapine carboxylic acid and quetiapine sulfoxide in the urine as the major metabolic urine compounds is shown in Table 5. Assuming 45 opportunities to determine the patient to be either positive or negative for Seroquel® dosing, the use of quetiapine carboxylic acid and quetiapine sulfoxide resulted in ˜96% correct identification of those taking the prescribed medicine. The data in Table 6 demonstrate the “normal” nature of the sample validity criteria (i.e., pH, specific gravity, and creatinine). Without the quetiapine carboxylic acid and quetiapine sulfoxide metabolites, only ˜58% were determined to be positive solely by the parent compound quetiapine and ˜67% were determined to be positive when using the parent compound quetiapine in conjunction with 7-hydroxy quetiapine. However, the use of all 4 analytes results in 100% correctly determined to be positive. Thus, use of quetiapine carboxylic acid and quetiapine sulfoxide as a urine biomarker at this low dose adds value to compliance monitoring for Seroquel®.

TABLE 5
Test Results from Patients Prescribed 50 mg/day Seroquel ® (all parent and
metabolite data reported in ng/mL).

| | | | | | | Quetiapine | | |
|---|---|---|---|---|---|---|---|---|
| | Specific | | Creatinine | | 7-Hydroxy | Carboxylic | Quetiapine | All 4 |
| Subject | Gravity | pH | (mg/dL) | Quetiapine | Quetiapine | Acid | Sulfoxide | mets |
| Q | 1.014 | 6.4 | 43.0 | 1271 | 1188 | >5000 | >5000 | + |
| S | 1.012 | 5.1 | 107.0 | ND | ND | 19 | ND | + |
| T | 1.013 | 6.9 | 162.9 | ND | ND | 6 | ND | + |
| U | 1.004 | 5.4 | 30.6 | 153 | 145 | >5000 | 1929 | + |
| V | 1.006 | 5.7 | 55.3 | 26 | 28 | 334 | 1551 | + |
| W | 1.003 | 7.1 | 31.6 | ND | ND | 5 | ND | + |
| X | 1.003 | 6.7 | 38.1 | ND | ND | 70 | 35 | + |
| Y | 1.011 | 8.2 | 81.6 | ND | 5 | 3272 | 153 | + |
| Z | 1.010 | 7.5 | 64.9 | 22 | 68 | >5000 | 398 | + |
| AA | 1.003 | 6.6 | 24.5 | ND | ND | 195 | 27 | + |
| AB | 1.021 | 5.0 | 230.7 | 268 | 49 | >5000 | 3410 | + |
| AC | 1.006 | 5.2 | 37.7 | 14 | 6 | 1570 | 244 | + |
| AD | 1.018 | 6.7 | 250.2 | 7 | ND | 443 | 269 | + |
| AF | 1.010 | 5.6 | 72.1 | ND | 9 | ND | ND | + |
| AG | 1.009 | 8.4 | 94.4 | ND | ND | 7 | ND | + |
| AH | 1.004 | 7.1 | 41.9 | 8 | 18 | >5000 | 156 | + |
| AI | 1.013 | 5.6 | 69.1 | 19 | 27 | 2892 | 879 | + |
| AJ | 1.014 | 5.8 | 114.4 | ND | ND | 5 | ND | + |
| AK | 1.022 | 6.3 | 188.1 | 62 | 75 | 4845 | 2693 | + |
| AL | 1.014 | 4.9 | 186.1 | ND | ND | 12 | ND | + |
| AM | 1.015 | 5.7 | 110.7 | 11 | 15 | 691 | 471 | + |
| AN | 1.006 | 7.4 | 146.6 | 35 | 108 | >5000 | 1437 | + |
| AO | 1.006 | 6.0 | 50.9 | 32 | ND | 419 | 230 | + |
| AP | 1.006 | 8.1 | 53.4 | ND | ND | 10 | ND | + |
| AQ | 1.019 | 5.8 | 253.6 | 1118 | 694 | >5000 | 740 | + |
| AR | 1.015 | 6.5 | 100.6 | ND | ND | 26 | ND | + |
| AS | 1.007 | 6.9 | 23.0 | 6 | 5 | 97 | 1061 | + |
| AT | 1.010 | 7.9 | 148.1 | 44 | 25 | 549 | 1313 | + |
| AU | 1.018 | 5.2 | 133.5 | 243 | 33 | ND | ND | + |
| AV | 1.013 | 6.7 | 110.1 | 86 | 8 | 1591 | 3226 | + |
| AX | 1.006 | 6.7 | 48.1 | ND | ND | 129 | 65 | + |
| AY | 1.016 | 6.3 | 217.6 | 169 | 58 | >5000 | >5000 | + |
| AZ | 1.016 | 5.5 | 92.3 | 361 | 298 | >5000 | >5000 | + |
| BA | 1.017 | 5.2 | 159.2 | 79 | 39 | 1328 | >5000 | + |
| BB | 1.006 | 7.7 | 49.6 | 38 | 24 | 2114 | 1521 | + |
| BC | 1.015 | 6.7 | 160.0 | 21 | 24 | >5000 | 306 | + |
| BD | 1.003 | 6.7 | 18.7 | ND | ND | 147 | 37 | + |
| BE | 1.006 | 6.4 | 83.7 | 221 | 75 | >5000 | 2675 | + |
| BF | 1.013 | 6.1 | 54.2 | ND | ND | 67 | 15 | + |
| BG | 1.008 | 7.8 | 40.8 | ND | 6 | 132 | 26 | + |
| BH | 1.015 | 5.4 | 108.8 | 23 | 10 | 415 | 129 | + |
| BI | 1.009 | 8.2 | 91.1 | ND | ND | 81 | 29 | + |
| BJ | 1.009 | 6.6 | 78.2 | ND | ND | 70 | ND | + |
| BL | 1.005 | 7.3 | 54.0 | ND | 12 | 637 | 113 | + |
| BM | 1.007 | 7.3 | 128.2 | 9 | 77 | 1136 | 709 | + |

ND: Indicates that the listed parent or metabolite compound was either not detected or detected below the established cut-off of 5 ng/mL indicating a negative result for that particular subject.

TABLE 6
Sample Validity and Urine Results Summary at 50 mg/day (all parent and
metabolite data reported in ng/mL).

| | | | | | | Quetiapine | |
|---|---|---|---|---|---|---|---|
| | Specific | | Creatinine | | 7-Hydroxy | Carboxylic | Quetiapine |
| | Gravity | pH | (mg/dL) | Quetiapine | Quetiapine | Acid | Sulfoxide |
| Average | 1.011 | 6.5 | 98.6 | 167 | 112 | 729 | 862 |
| Standard | 0.005 | 1.0 | 62.5 | 312 | 246 | 1119 | 1002 |
| Deviation | | | | | | | |
| Maximum | 1.022 | 8.4 | 253.6 | 1271 | 1188 | >5000 | >5000 |
| Value | | | | | | | |
| Median | 1.010 | 6.6 | 83.7 | 36 | 28 | 171 | 352 |
| Value | | | | | | | |
| Minimum | 1.003 | 4.9 | 18.7 | 6 | 5 | 5 | 15 |
| Value | | | | | | | |

### Example 4

The urine of 75 patients who were prescribed 100 mg of Seroquel® (quetiapine) was tested for compliance (Table 7). 100 μL of each patient specimen was diluted with 50 μL of 1.6 μg/mL of quetiapine-D8 (internal standard) in methanol and 350 μL of 0.1% formic acid. Samples were subsequently vortexed and centrifuged prior to the injection of 5 μL on the QQQ.

The preponderance of quetiapine carboxylic acid and quetiapine sulfoxide in the urine as the major metabolic urine compounds is shown in Table 7. Assuming 75 opportunities to determine the patient to be either positive or negative for Seroquel® dosing, the use of quetiapine carboxylic acid and quetiapine sulfoxide resulted in ˜99% correct identification of those taking the prescribed medicine. The data in Table 8 demonstrate the “normal” nature of the sample validity criteria (i.e., pH, specific gravity, and creatinine). Without the quetiapine carboxylic acid and quetiapine sulfoxide metabolites, only ˜76% were determined to be positive solely by the parent compound quetiapine and ˜81% were determined to be positive when using the parent compound quetiapine in conjunction with 7-hydroxy quetiapine. However, again, the use of all 4 analytes correctly determines 100% of prescribed patients at this does. Thus, use of quetiapine carboxylic acid and quetiapine sulfoxide as a urine biomarker at this dose does add value to compliance monitoring for Seroquel®.

TABLE 7
Test Results from Patients Prescribed 100 mg/day Seroquel ® (all parent and
metabolite data reported in ng/mL).

| | | | | | | Quetiapine | | |
|---|---|---|---|---|---|---|---|---|
| | Specific | | Creatinine | | 7-Hydroxy | Carboxylic | Quetiapine | All 4 |
| Subject | Gravity | pH | (mg/dL) | Quetiapine | Quetiapine | Acid | Sulfoxide | mets |
| BN | 1.016 | 4.6 | 245.5 | >5000 | 1286 | >5000 | >5000 | + |
| BO | 1.011 | 6.4 | 120.3 | ND | ND | 98 | 11 | + |
| BP | 1.020 | 5.0 | 227.0 | 191 | 70 | >5000 | 2611 | + |
| BQ | 1.005 | 5.4 | 52.7 | 331 | 40 | >5000 | 843 | + |
| BR | 1.006 | 5.6 | 62.0 | 267 | 125 | >5000 | 2305 | + |
| BS | 1.015 | 5.9 | 209.2 | 428 | 420 | >5000 | >5000 | + |
| BT | 1.012 | 6.2 | 82.7 | 14 | 7 | 2321 | 442 | + |
| BU | 1.018 | 5.9 | 233.4 | ND | ND | 26 | 17 | + |
| BV | 1.012 | 5.6 | 103.3 | ND | ND | 22 | 7 | + |
| BW | 1.011 | 6.9 | 172.2 | 17 | 11 | >5000 | 765 | + |
| BX | 1.014 | 5.9 | 187.3 | 15 | 18 | 1554 | 659 | + |
| BY | 1.005 | 8.0 | 107.5 | 17 | 51 | >5000 | 2143 | + |
| BZ | 1.014 | 6.2 | 185.7 | 199 | 183 | >5000 | >5000 | + |
| CA | 1.009 | 5.7 | 58.0 | ND | ND | 47 | 8 | + |
| CB | 1.003 | 7.9 | 30.4 | ND | 6 | ND | ND | + |
| CC | 1.011 | 6.7 | 69.0 | 59 | 171 | >5000 | 1918 | + |
| CD | 1.018 | 5.5 | 221.9 | 138 | 89 | >5000 | 4782 | + |
| CE | 1.009 | 7.7 | 133.8 | ND | 8 | 1689 | 207 | + |
| CF | 1.014 | 6.0 | 126.4 | 233 | 1996 | >5000 | >5000 | + |
| CG | 1.013 | 5.8 | 90.6 | ND | 357 | >5000 | >5000 | + |
| CH | 1.018 | 6.1 | 203.1 | ND | 258 | >5000 | >5000 | + |
| CI | 1.008 | 7.1 | 50.3 | ND | ND | >5000 | 130 | + |
| CJ | 1.005 | 7.0 | 74.5 | 26 | 10 | >5000 | 265 | + |
| CK | 1.012 | 5.9 | 138.1 | 39 | ND | 1123 | 1100 | + |
| CL | 1.009 | 4.9 | 105.1 | 77 | 71 | 2816 | 2998 | + |
| CM | 1.012 | 6.3 | 128.5 | ND | ND | 73 | 7 | + |
| CN | 1.004 | 7.0 | 27.8 | 29 | 12 | 3065 | 420 | + |
| CO | 1.009 | 5.2 | 75.4 | 7 | 5 | 834 | 185 | + |
| CP | 1.005 | 6.3 | 59.4 | 13 | 24 | 515 | 349 | + |
| CQ | 1.018 | 5.6 | 193.4 | 194 | 230 | >5000 | >5000 | + |
| CR | 1.008 | 7.0 | 97.1 | 26 | 34 | >5000 | 506 | + |
| CS | 1.009 | 7.3 | 127.4 | 135 | 31 | >5000 | 1722 | + |
| CT | 1.013 | 6.8 | 161.5 | ND | ND | 9 | ND | + |
| CU | 1.014 | 5.4 | 126.3 | 412 | 106 | 3639 | >5000 | + |
| CV | 1.019 | 5.9 | 377.5 | 438 | 188 | >5000 | >5000 | + |
| CW | 1.013 | 5.2 | 193.5 | ND | ND | 8 | ND | + |
| CX | 1.011 | 7.0 | 153.5 | 43 | 94 | 2444 | >5000 | + |
| CY | 1.009 | 7.9 | 146.9 | 69 | 27 | 4405 | ND | + |
| CZ | 1.011 | 7.6 | 101.6 | 9 | 21 | 4491 | 1152 | + |
| DA | 1.010 | 7.2 | 86.6 | ND | ND | 16 | 176 | + |
| DB | 1.015 | 5.3 | 172.0 | 169 | 53 | >5000 | >5000 | + |
| DC | 1.011 | 6.9 | 66.0 | 268 | 95 | >5000 | >5000 | + |
| DD | 1.014 | 7.5 | 121.9 | 305 | 85 | >5000 | 3044 | + |
| DE | 1.009 | 6.2 | 108.7 | 265 | 150 | >5000 | >5000 | + |
| DF | 1.009 | 4.6 | 151.6 | 803 | 159 | 2927 | >5000 | + |
| DG | 1.009 | 5.2 | 87.9 | 19 | 20 | 971 | 1621 | + |
| DH | 1.016 | 5.1 | 192.3 | 314 | 265 | >5000 | >5000 | + |
| DI | 1.016 | 6.2 | 150.2 | 136 | 65 | >5000 | >5000 | + |
| DJ | 1.016 | 5.6 | 238.1 | 234 | 77 | >5000 | >5000 | + |
| DK | 1.010 | 7.1 | 91.9 | 6 | 5 | 1416 | 1862 | + |
| DL | 1.013 | 7.3 | 122.3 | 8 | ND | 102 | 124 | + |
| DM | 1.012 | 7.6 | 107.1 | 6 | ND | >5000 | 901 | + |
| DN | 1.006 | 6.5 | 39.7 | 43 | 24 | >5000 | >5000 | + |
| DO | 1.019 | 4.8 | 193.7 | 28 | 50 | 284 | >5000 | + |
| DP | 1.013 | 5.4 | 175.4 | 22 | 53 | 336 | >5000 | + |
| DQ | 1.010 | 5.7 | 86.7 | 49 | 100 | 2802 | >5000 | + |
| DR | 1.008 | 5.6 | 42.6 | ND | ND | 119 | 192 | + |
| DS | 1.019 | 5.4 | 172.2 | 9 | 49 | 1321 | 1131 | + |
| DT | 1.011 | 6.4 | 130.5 | 76 | 482 | >5000 | ND | + |
| DU | 1.012 | 6.0 | 230.3 | 328 | 183 | ND | >5000 | + |
| DV | 1.006 | 7.3 | 64.2 | 131 | 338 | >5000 | >5000 | + |
| DW | 1.010 | 5.8 | 127.8 | 328 | 145 | >5000 | >5000 | + |
| DX | 1.018 | 5.7 | 257.1 | ND | ND | 15 | ND | + |
| DY | 1.008 | 5.3 | 86.8 | 91 | 14 | 866 | 528 | + |
| DZ | 1.009 | 5.4 | 86.4 | 52 | 24 | 407 | 738 | + |
| EA | 1.004 | 5.8 | 25.3 | ND | ND | 28 | 13 | + |
| EB | 1.017 | 6.5 | 180.9 | 10 | 7 | 1612 | 220 | + |
| EC | 1.019 | 5.9 | 149.1 | 230 | 27 | >5000 | 1256 | + |
| ED | 1.013 | 6.1 | 113.8 | ND | ND | 10 | ND | + |
| EE | 1.011 | 6.6 | 133.9 | 312 | 301 | >5000 | >5000 | + |
| EF | 1.021 | 5.3 | 169.8 | ND | ND | 46 | 9 | + |
| EG | 1.005 | 7.5 | 52.0 | 22 | 54 | 666 | 980 | + |
| EH | 1.005 | 7.0 | 23.4 | 26 | 37 | 1525 | 780 | + |
| EI | 1.027 | 5.8 | 372.6 | 49 | 39 | 2168 | 326 | + |
| EJ | 1.017 | 5.8 | 188.5 | 53 | 28 | 2602 | 742 | + |

ND: Indicates that the listed parent or metabolite compound was either not detected or detected below the established cut-off of 5 ng/mL indicating a negative result for that particular subject.

TABLE 8
Sample Validity and Urine Results Summary at 100 mg/day (all parent and
metabolite data reported in ng/mL).

| | | | | | | Quetiapine | |
|---|---|---|---|---|---|---|---|
| | Specific | | Creatinine | | 7-Hydroxy | Carboxylic | Quetiapine |
| | Gravity | pH | (mg/dL) | Quetiapine | Quetiapine | Acid | Sulfoxide |
| Average | 1.012 | 6.2 | 134.1 | 140 | 153 | 1235 | 935 |
| Standard | 0.005 | 0.9 | 71.2 | 155 | 309 | 1295 | 1023 |
| Deviation | | | | | | | |
| Maximum | 1.027 | 8.0 | 377.5 | >5000 | 1996 | >5000 | >5000 |
| Value | | | | | | | |
| Median | 1.011 | 6.0 | 126.4 | 64 | 54 | 850 | 659 |
| Value | | | | | | | |
| Minimum | 1.003 | 4.6 | 23.4 | 6 | 5 | 8 | 7 |
| Value | | | | | | | |

### Example 5

The urine of 11 patients who were prescribed 150 mg of Seroquel® (quetiapine) was tested for compliance (Table 9). 100 μL of each patient specimen was diluted with 50 μL of 1.6 μg/mL of quetiapine-D8 (internal standard) in methanol and 350 μL of 0.1% formic acid. Samples were subsequently vortexed and centrifuged prior to the injection of 5 μL on the QQQ. Each patient sample was analyzed twice to ensure accuracy.

The preponderance of quetiapine carboxylic acid and quetiapine sulfoxide in the urine as the major metabolic urine compounds is shown in Table 9. Assuming 12 opportunities to determine the patient to be either positive or negative for Seroquel® dosing, the use of quetiapine carboxylic acid and quetiapine sulfoxide resulted in ˜100% correct identification of those taking the prescribed medicine. The data in Table 10 demonstrate the “normal” nature of the sample validity criteria (i.e., pH, specific gravity, and creatinine). Without the quetiapine carboxylic acid and quetiapine sulfoxide metabolites, only ˜82% were determined to be positive solely by the parent compound quetiapine and ˜82% were determined to be positive when using the parent compound quetiapine in conjunction with 7-hydroxy quetiapine. However, again, the use of all 4 analytes correctly determines 100% of prescribed patients at this does. Thus, use of quetiapine carboxylic acid and quetiapine sulfoxide as a urine biomarker at this dose does add value to compliance monitoring for Seroquel®.

TABLE 9
Test Results from Patients Prescribed 150 mg/day Seroquel ® (all parent and
metabolite data reported in ng/mL).

| | | | | | | Quetiapine | | |
|---|---|---|---|---|---|---|---|---|
| | Specific | | Creatinine | | 7-Hydroxy | Carboxylic | Quetiapine | All 4 |
| Subject | Gravity | pH | (mg/dL) | Quetiapine | Quetiapine | Acid | Sulfoxide | mets |
| EK | 1.011 | 6.9 | 165.9 | 36 | 86 | >5000 | 813 | + |
| EL | 1.016 | 6.5 | 151.2 | 20 | 150 | 4611 | 834 | + |
| EM | 1.004 | 7.7 | 25.4 | 12 | 6 | 2296 | 356 | + |
| EN | 1.009 | 7.5 | 85.7 | 79 | 48 | >5000 | 4511 | + |
| EO | 1.020 | 5.7 | 170.9 | 666 | 152 | >5000 | >5000 | + |
| EP | 1.005 | 4.9 | 43.9 | 12 | 37 | 511 | 480 | + |
| EQ | 1.005 | 7.3 | 104.4 | 120 | 121 | >5000 | >5000 | + |
| ER | 1.002 | 6.9 | 17.3 | ND | ND | 80 | 30 | + |
| ES | 1.013 | 7.2 | 150.6 | 48 | ND | 4136 | 610 | + |
| EV | 1.009 | 5.8 | 78.3 | ND | ND | 23 | 28 | + |
| EW | 1.006 | 7.9 | 64.8 | 8 | 16 | 4653 | 451 | + |

ND: Indicates that the listed parent or metabolite compound was either not detected or detected below the established cut-off of 5 ng/mL indicating a negative result for that particular subject.

TABLE 10
Sample Validity and Urine Results Summary at 150 mg/day (all parent and
metabolite data reported in ng/mL).

| | | | | | | Quetiapine | |
|---|---|---|---|---|---|---|---|
| | Specific | | Creatinine | | 7-Hydroxy | Carboxylic | Quetiapine |
| | Gravity | pH | (mg/dL) | Quetiapine | Quetiapine | Acid | Sulfoxide |
| Average | 1.009 | 6.8 | 96.2 | 111 | 77 | 2330 | 901 |
| Standard | 0.005 | 0.9 | 53.8 | 199 | 55 | 1984 | 1305 |
| Deviation | | | | | | | |
| Maximum | 1.020 | 7.9 | 170.9 | 666 | 152 | >5000 | >5000 |
| Value | | | | | | | |
| Median | 1.009 | 6.9 | 85.7 | 36 | 67 | 2296 | 480 |
| Value | | | | | | | |
| Minimum | 1.002 | 4.9 | 17.3 | 8 | 6 | 23 | 28 |
| Value | | | | | | | |

### Example 6

The urine of 59 patients who were prescribed 200 mg of Seroquel® (quetiapine) was tested for compliance (Table 11). 100 μL of each patient specimen was diluted with 50 μL of 1.6 μg/mL of quetiapine-D8 (internal standard) in methanol and 350 μL of 0.1% formic acid. Samples were subsequently vortexed and centrifuged prior to the injection of 5 μL on the QQQ.

The preponderance of quetiapine carboxylic acid and quetiapine sulfoxide in the urine as the major metabolic urine compounds is shown in Table 11. Assuming 59 opportunities to determine the patient to be either positive or negative for Seroquel® dosing, the use of quetiapine carboxylic acid and quetiapine sulfoxide resulted in ˜100% correct identification of those taking the prescribed medicine. The data in Table 11 demonstrate the “normal” nature of the sample validity criteria (i.e., pH, specific gravity, and creatinine). Without the quetiapine carboxylic acid and quetiapine sulfoxide metabolites, only ˜75% were determined to be positive solely by the parent compound quetiapine and ˜83% were determined to be positive when using the parent compound quetiapine in conjunction with 7-hydroxy quetiapine. However, when all 4 analytes are used to assess adherence, the result is 100% as per prescription. Thus, use of quetiapine carboxylic acid and quetiapine sulfoxide as a urine biomarker at this dose does add value to compliance monitoring for Seroquel®.

TABLE 11
Test Results from Patients Prescribed 200 mg/day Seroquel ® (all parent
and metabolite data reported in ng/mL).

| | | | | | | Quetiapine | | |
|---|---|---|---|---|---|---|---|---|
| | Specific | | Creatinine | | 7-Hydroxy | Carboxylic | Quetiapine | All 4 |
| Subject | Gravity | pH | (mg/dL) | Quetiapine | Quetiapine | Acid | Sulfoxide | mets |
| EX | 1.003 | 5.1 | 42.7 | 6 | ND | 76 | 74 | + |
| EY | 1.005 | 6.4 | 74.0 | 17 | 243 | >5000 | 696 | + |
| FA | 1.012 | 5.2 | 143.3 | 182 | 34 | 1179 | >5000 | + |
| FB | 1.010 | 7.8 | 219.4 | 100 | 178 | >5000 | >5000 | + |
| FC | 1.007 | 7.7 | 91.6 | 368 | 236 | >5000 | 3376 | + |
| FE | 1.009 | 6.9 | 164.9 | 67 | 335 | >5000 | 4792 | + |
| FF | 1.010 | 5.7 | 150.5 | ND | ND | 22 | ND | + |
| FG | 1.007 | 6.3 | 134.7 | 74 | 336 | >5000 | 4490 | + |
| FH | 1.017 | 5.5 | 275.9 | 20 | 21 | 1305 | 797 | + |
| FI | 1.011 | 6.1 | 144.7 | 538 | 430 | >5000 | >5000 | + |
| FJ | 1.011 | 6.4 | 115.2 | 143 | 2066 | >5000 | >5000 | + |
| FK | 1.015 | 6.7 | 98.9 | ND | 37 | 11 | ND | + |
| FL | 1.013 | 6.3 | 175.9 | 2322 | 3844 | >5000 | >5000 | + |
| FM | 1.007 | 7.5 | 121.0 | ND | 46 | 3224 | 95 | + |
| FN | 1.005 | 7.6 | 126.1 | 411 | 293 | >5000 | >5000 | + |
| FO | 1.010 | 5.8 | 65.6 | 9 | 81 | 947 | 730 | + |
| FP | 1.013 | 6.2 | 116.2 | 19 | 102 | >5000 | 1058 | + |
| FQ | 1.005 | 7.4 | 60.2 | 5 | 39 | 1781 | 543 | + |
| FR | 1.019 | 5.6 | 177.5 | ND | 20 | 383 | 368 | + |
| FS | 1.002 | 6.7 | 29.6 | ND | ND | 25 | ND | + |
| FT | 1.006 | 5.4 | 27.9 | 73 | 10 | 1355 | 414 | + |
| FU | 1.012 | 6.9 | 318.9 | ND | 18 | >5000 | >5000 | + |
| FV | 1.012 | 6.5 | 192.1 | 208 | 262 | >5000 | >5000 | + |
| FW | 1.017 | 4.9 | 326.9 | 315 | 29 | >5000 | >5000 | + |
| FX | 1.006 | 6.8 | 66.8 | 97 | 5 | >5000 | 2971 | + |
| GA | 1.013 | 6.1 | 123.8 | 22 | 13 | 1378 | 361 | + |
| GB | 1.009 | 7.0 | 110.1 | ND | ND | 179 | ND | + |
| GC | 1.005 | 5.1 | 56.7 | 9 | 9 | 1302 | 143 | + |
| GD | 1.006 | 5.9 | 37.4 | 261 | 178 | 4523 | >5000 | + |
| GE | 1.004 | 8.6 | 38.2 | 64 | 51 | 2163 | 2289 | + |
| GF | 1.016 | 6.2 | 128.8 | 187 | 262 | >5000 | >5000 | + |
| GG | 1.015 | 5.7 | 185.5 | 222 | 116 | >5000 | >5000 | + |
| GH | 1.009 | 6.6 | 150.6 | 44 | 86 | >5000 | 1209 | + |
| GI | 1.018 | 7.0 | 204.9 | 62 | 168 | >5000 | 2195 | + |
| GJ | 1.018 | 5.8 | 229.0 | 159 | 9 | ND | >5000 | + |
| GK | 1.012 | 6.2 | 173.0 | ND | ND | 5 | ND | + |
| GM | 1.017 | 5.8 | 110.0 | 198 | 151 | >5000 | >5000 | + |
| GN | 1.005 | 6.2 | 77.1 | 33 | 44 | 3801 | >5000 | + |
| GO | 1.016 | 5.6 | 160.2 | 286 | 88 | >5000 | >5000 | + |
| GQ | 1.014 | 6.5 | 156.8 | 14 | 19 | >5000 | 946 | + |
| GR | 1.009 | 5.2 | 141.0 | 277 | 100 | 504 | 3488 | + |
| GS | 1.015 | 5.5 | 95.9 | 26 | ND | 461 | 1280 | + |
| GT | 1.018 | 5.2 | 143.2 | 229 | 69 | >5000 | >5000 | + |
| GU | 1.005 | 7.1 | 50.6 | ND | 16 | 3620 | 27 | + |
| GV | 1.021 | 5.2 | 221.7 | ND | ND | 5 | 15 | + |
| GW | 1.006 | 7.8 | 73.4 | 65 | 34 | >5000 | >5000 | + |
| GX | 1.014 | 6.3 | 201.9 | 24 | 163 | >5000 | >5000 | + |
| GY | 1.020 | 6.0 | 208.0 | 69 | 252 | >5000 | >5000 | + |
| GZ | 1.014 | 5.6 | 129.4 | 55 | 138 | 1197 | >5000 | + |
| HA | 1.017 | 6.2 | 314.6 | ND | ND | 5 | ND | + |
| HB | 1.010 | 7.4 | 127.0 | 13 | 34 | 3265 | 343 | + |
| HC | 1.006 | 7.3 | 56.1 | ND | ND | 12 | ND | + |
| HD | 1.007 | 8.1 | 105.2 | 19 | 25 | 4501 | 321 | + |
| HE | 1.019 | 5.5 | 275.3 | ND | ND | 9 | ND | + |
| HF | 1.006 | 6.0 | 29.0 | 139 | 110 | >5000 | 2954 | + |
| HG | 1.009 | 5.6 | 83.5 | ND | ND | 86 | 11 | + |
| HH | 1.018 | 5.2 | 235.3 | ND | ND | 23 | ND | + |
| HI | 1.012 | 5.4 | 89.7 | 387 | 153 | >5000 | 4234 | + |
| HJ | 1.003 | 6.8 | 21.9 | 196 | 37 | >5000 | 1261 | + |

ND: Indicates that the listed parent or metabolite compound was either not detected or detected below the established cut-off of 5 ng/mL indicating a negative result for that particular subject.

TABLE 12
Sample Validity and Urine Results Summary at 200 mg/day (all parent and
metabolite data reported in ng/mL).

| | | | | | | Quetiapine | |
|---|---|---|---|---|---|---|---|
| | Specific | | Creatinine | | 7-Hydroxy | Carboxylic | Quetiapine |
| | Gravity | pH | (mg/dL) | Quetiapine | Quetiapine | Acid | Sulfoxide |
| Average | 1.011 | 6.3 | 135.7 | 183 | 234 | 1245 | 1430 |
| Standard | 0.005 | 0.9 | 75.5 | 351 | 611 | 1440 | 1465 |
| Deviation | | | | | | | |
| Maximum | 1.021 | 8.6 | 326.9 | 2322 | 3844 | >5000 | >5000 |
| Value | | | | | | | |
| Median | 1.011 | 6.2 | 127.0 | 73 | 86 | 726 | 797 |
| Value | | | | | | | |
| Minimum | 1.002 | 4.9 | 21.9 | 5 | 5 | 5 | 11 |
| Value | | | | | | | |

### Example 7

The urine of 59 patients who were prescribed 300 mg of Seroquel® (quetiapine) was tested for compliance (Table 13). 100 μL of each patient specimen was diluted with 50 μL of 1.6 μg/mL of quetiapine-D8 (internal standard) in methanol and 350 μL of 0.1% formic acid. Samples were subsequently vortexed and centrifuged prior to the injection of 5 μL on the QQQ.

The preponderance of quetiapine carboxylic acid and quetiapine sulfoxide in the urine as the major metabolic urine compounds is shown in Table 13. Assuming 58 opportunities to determine the patient to be either positive or negative for Seroquel® dosing, the use of quetiapine carboxylic acid and quetiapine sulfoxide resulted in ˜100% correct identification of those taking the prescribed medicine. The data in Table 14 demonstrate the “normal” nature of the sample validity criteria (i.e., pH, specific gravity, and creatinine). Without the quetiapine carboxylic acid and quetiapine sulfoxide metabolites, ˜86% were determined to be positive solely by the parent compound quetiapine and ˜92% were determined to be positive when using the parent compound quetiapine in conjunction with 7-hydroxy quetiapine. Use of all 4 analytes results in 100% identification of those taking the prescribed medicine. Thus, use of quetiapine carboxylic acid and quetiapine sulfoxide as a urine biomarker at this dose does add value to compliance monitoring for Seroquel®.

TABLE 13
Test Results from Patients Prescribed 300 mg/day Seroquel ® (all parent
and metabolite data reported in ng/mL).

| | | | | | | Quetiapine | | |
|---|---|---|---|---|---|---|---|---|
| | Specific | | Creatinine | | 7-Hydroxy | Carboxylic | Quetiapine | All 4 |
| Subject | Gravity | pH | (mg/dL) | Quetiapine | Quetiapine | Acid | Sulfoxide | mets |
| HI | 1.019 | 5.4 | 266.9 | 978 | 809 | >5000 | >5000 | + |
| HJ | 1.007 | 5.9 | 103.0 | 42 | 200 | >5000 | 2356 | + |
| HK | 1.012 | 5.7 | 110.8 | ND | ND | 63 | ND | + |
| HL | 1.014 | 7.5 | 129.7 | ND | ND | 122 | 35 | + |
| HM | 1.017 | 5.7 | 109.3 | 19 | 21 | 1048 | 489 | + |
| HN | 1.006 | 7.0 | 45.5 | 39 | 795 | >5000 | >5000 | + |
| HO | 1.009 | 6.2 | 97.6 | 223 | 273 | >5000 | 4046 | + |
| HP | 1.008 | 6.9 | 56.4 | 8 | ND | 808 | 181 | + |
| HQ | 1.010 | 6.3 | 118.8 | 269 | 133 | >5000 | >5000 | + |
| HR | 1.013 | 5.4 | 99.7 | 4574 | >5000 | >5000 | >5000 | + |
| HS | 1.018 | 5.8 | 176.7 | 755 | >5000 | >5000 | >5000 | + |
| HT | 1.003 | 6.6 | 36.9 | 44 | 113 | >5000 | 1612 | + |
| HU | 1.007 | 6.2 | 44.8 | 42 | 73 | >5000 | 1330 | + |
| HV | 1.012 | 5.8 | 95.7 | 200 | 149 | >5000 | >5000 | + |
| HW | 1.010 | 5.4 | 126.2 | 462 | 504 | >5000 | >5000 | + |
| HX | 1.024 | 8.9 | 46.3 | >5000 | >5000 | >5000 | >5000 | + |
| HY | 1.018 | 6.0 | 139.4 | 917 | 1961 | >5000 | >5000 | + |
| HZ | 1.019 | 5.7 | 112.9 | 196 | 370 | >5000 | >5000 | + |
| IA | 1.006 | 5.2 | 70.9 | 80 | 100 | >5000 | 1909 | + |
| IB | 1.004 | 7.3 | 51.2 | 121 | 170 | >5000 | 4538 | + |
| IC | 1.004 | 8.1 | 84.1 | 18 | 69 | >5000 | 1414 | + |
| ID | 1.007 | 6.1 | 68.2 | 394 | 686 | >5000 | >5000 | + |
| IE | 1.009 | 6.4 | 93.1 | ND | 52 | 636 | 367 | + |
| IF | 1.011 | 6.2 | 110.3 | ND | 200 | >5000 | >5000 | + |
| IG | 1.014 | 5.5 | 128.3 | ND | ND | 3610 | 858 | + |
| IH | 1.014 | 6.0 | 255.1 | 361 | 63 | >5000 | >5000 | + |
| II | 1.007 | 7.7 | 84.2 | 1237 | 84 | >5000 | 2043 | + |
| IJ | 1.012 | 7.0 | 161.0 | 569 | 211 | >5000 | >5000 | + |
| IK | 1.008 | 7.7 | 93.0 | 873 | 2034 | >5000 | >5000 | + |
| IL | 1.004 | 8.1 | 35.9 | 29 | 40 | >5000 | 339 | + |
| IM | 1.011 | 6.6 | 109.4 | 347 | 379 | >5000 | >5000 | + |
| IN | 1.006 | 5.7 | 26.5 | 177 | 90 | >5000 | 1390 | + |
| IP | 1.010 | 7.6 | 121.4 | ND | 9 | 994 | 95 | + |
| IQ | 1.006 | 7.3 | 56.1 | 55 | 313 | >5000 | 4135 | + |
| IS | 1.010 | 6.5 | 136.4 | 38 | 10 | >5000 | 1662 | + |
| IT | 1.003 | 7.0 | 20.7 | 9 | 8 | 3008 | 3343 | + |
| IU | 1.022 | 5.3 | 288.4 | 486 | 280 | >5000 | >5000 | + |
| IV | 1.005 | 7.8 | 25.0 | 26 | 67 | 4772 | >5000 | + |
| IW | 1.019 | 6.2 | 311.4 | 740 | 418 | >5000 | >5000 | + |
| IX | 1.012 | 5.6 | 120.2 | 205 | 46 | >5000 | >5000 | + |
| IY | 1.022 | 5.6 | 178.0 | 852 | 191 | >5000 | >5000 | + |
| IZ | 1.009 | 6.3 | 180.3 | ND | ND | 114 | 56 | + |
| JA | 1.011 | 6.8 | 109.5 | 7 | 50 | 1517 | 520 | + |
| JB | 1.017 | 6.9 | 183.7 | >5000 | 1326 | >5000 | >5000 | + |
| JC | 1.011 | 7.2 | 104.7 | 31 | 9 | 3973 | 2387 | + |
| JD | 1.004 | 7.9 | 83.3 | 10 | 18 | >5000 | 2610 | + |
| JE | 1.018 | 5.7 | 186.7 | 2138 | 1006 | >5000 | >5000 | + |
| JF | 1.006 | 5.7 | 90.1 | 243 | 287 | >5000 | >5000 | + |
| JG | 1.021 | 5.8 | 373.0 | 127 | 73 | 3152 | 3093 | + |
| JI | 1.016 | 7.1 | 83.6 | 16 | 24 | 1648 | 376 | + |
| JJ | 1.004 | 5.3 | 34.3 | 530 | 169 | >5000 | >5000 | + |
| JK | 1.007 | 7.3 | 73.2 | 17 | 28 | >5000 | 447 | + |
| JL | 1.011 | 5.5 | 114.4 | 442 | 101 | >5000 | >5000 | + |
| JM | 1.008 | 6.4 | 82.0 | 109 | 100 | >5000 | 3087 | + |
| JN | 1.016 | 5.3 | 111.8 | ND | ND | 126 | 17 | + |
| JO | 1.011 | 5.8 | 150.8 | 695 | 410 | >5000 | >5000 | + |
| JP | 1.006 | 8.4 | 109.9 | 52 | 225 | >5000 | >5000 | + |
| JQ | 1.014 | 6.3 | 166.0 | 67 | 29 | >5000 | 947 | + |
| JR | 1.010 | 6.2 | 84.3 | 56 | 25 | 935 | 686 | + |

ND: Indicates that the listed parent or metabolite compound was either not detected or detected below the established cut-off of 5 ng/mL indicating a negative result for that particular subject.

TABLE 14
Sample Validity and Urine Results Summary at 300 mg/day (all parent and
metabolite data reported in ng/mL).

| | | | | | | Quetiapine | |
|---|---|---|---|---|---|---|---|
| | Specific | | Creatinine | | 7-Hydroxy | Carboxylic | Quetiapine |
| | Gravity | pH | (mg/dL) | Quetiapine | Quetiapine | Acid | Sulfoxide |
| Average | 1.011 | 6.5 | 116.4 | 407 | 296 | 1658 | 1546 |
| Standard | 0.005 | 0.9 | 70.3 | 727 | 441 | 1493 | 1327 |
| Deviation | | | | | | | |
| Maximum | 1.024 | 8.9 | 373.0 | >5000 | >5000 | >5000 | >5000 |
| Value | | | | | | | |
| Median | 1.010 | 6.2 | 109.3 | 177 | 123 | 1021 | 1360 |
| Value | | | | | | | |
| Minimum | 1.003 | 5.2 | 20.7 | 7 | 8 | 63 | 17 |
| Value | | | | | | | |

### Example 8

The urine of 41 patients who were prescribed 400 mg of Seroquel® (quetiapine) was tested for compliance (Table 15). 100 μL of each patient specimen was diluted with 50 μL of 1.6 μg/mL of quetiapine-D8 (internal standard) in methanol and 350 μL of 0.1% formic acid. Samples were subsequently vortexed and centrifuged prior to the injection of 5 μL on the QQQ.

The preponderance of quetiapine carboxylic acid and quetiapine sulfoxide in the urine as the major metabolic urine compounds is shown in Table 15. Assuming 41 opportunities to determine the patient to be either positive or negative for Seroquel® dosing, the use of quetiapine carboxylic acid and quetiapine sulfoxide resulted in ˜100% correct identification of those taking the prescribed medicine. The data in Table 16 demonstrate the “normal” nature of the sample validity criteria (i.e., pH, specific gravity, and creatinine). Without the quetiapine carboxylic acid and quetiapine sulfoxide metabolites, ˜73% were determined to be positive solely by the parent compound quetiapine and ˜80% were determined to be positive when using the parent compound quetiapine in conjunction with 7-hydroxy quetiapine. Using all 4 analytes resulted in 100% correct identification of those taking the prescribed medicine. Thus, use of quetiapine carboxylic acid and quetiapine sulfoxide as a urine biomarker at this dose does add value to compliance monitoring for Seroquel®.

TABLE 15
Test Results from Patients Prescribed 400 mg/day Seroquel ® (all parent
and metabolite data reported in ng/mL).

| | | | | | | Quetiapine | | |
|---|---|---|---|---|---|---|---|---|
| | Specific | | Creatinine | | 7-Hydroxy | Carboxylic | Quetiapine | All 4 |
| Subject | Gravity | pH | (mg/dL) | Quetiapine | Quetiapine | Acid | Sulfoxide | mets |
| JS | 1.008 | 5.7 | 78.0 | 128 | 394 | >5000 | >5000 | + |
| JT | 1.007 | 7.7 | 148.9 | 29 | 376 | >5000 | >5000 | + |
| JU | 1.009 | 5.6 | 87.1 | ND | ND | 9 | ND | + |
| JW | 1.010 | 5.8 | 92.8 | ND | ND | 496 | 47 | + |
| JX | 1.011 | 5.8 | 144.7 | 560 | 876 | >5000 | >5000 | + |
| JY | 1.008 | 7.6 | 98.8 | 77 | 324 | >5000 | 4484 | + |
| JZ | 1.011 | 5.7 | 59.8 | 73 | 58 | >5000 | 3198 | + |
| KA | 1.008 | 7.5 | 60.3 | ND | 29 | 935 | 46 | + |
| KB | 1.006 | 7.7 | 113.8 | 45 | 235 | >5000 | >5000 | + |
| KC | 1.011 | 6.7 | 166.7 | ND | ND | 13 | ND | + |
| KD | 1.007 | 7.3 | 26.3 | 42 | 86 | >5000 | 2943 | + |
| KE | 1.014 | 5.6 | 168.1 | ND | ND | 328 | 5 | + |
| KG | 1.003 | 7.3 | 48.7 | 304 | 580 | >5000 | >5000 | + |
| KH | 1.015 | 6.3 | 249.2 | 1137 | 958 | >5000 | >5000 | + |
| KI | 1.015 | 5.5 | 206.3 | 104 | 133 | >5000 | 4464 | + |
| KJ | 1.017 | 5.8 | 95.9 | ND | ND | 28 | 12 | + |
| KK | 1.013 | 6.4 | 86.3 | 11 | 8 | 519 | 874 | + |
| KL | 1.011 | 6.6 | 96.7 | ND | ND | 24 | ND | + |
| KM | 1.008 | 7.8 | 48.9 | ND | ND | 10 | 6 | + |
| KN | 1.017 | 6.0 | 285.5 | ND | 217 | >5000 | >5000 | + |
| KQ | 1.005 | 6.9 | 44.1 | 101 | 64 | >5000 | 2027 | + |
| KR | 1.002 | 6.5 | 23.8 | ND | 10 | 1179 | 10 | + |
| KS | 1.015 | 6.0 | 205.8 | 138 | 159 | >5000 | >5000 | + |
| KT | 1.012 | 6.0 | 153.3 | 1616 | 311 | >5000 | >5000 | + |
| KU | 1.008 | 5.9 | 50.8 | 763 | 234 | >5000 | >5000 | + |
| KV | 1.018 | 5.7 | 288.7 | 120 | 39 | >5000 | >5000 | + |
| KW | 1.004 | 7.6 | 82.3 | 7 | 32 | 523 | >5000 | + |
| KX | 1.014 | 7.1 | 119.6 | ND | ND | 70 | 136 | + |
| KZ | 1.012 | 5.7 | 213.3 | 112 | 148 | >5000 | >5000 | + |
| LA | 1.006 | 5.7 | 45.4 | 159 | 341 | >5000 | >5000 | + |
| LB | 1.010 | 8.2 | 67.0 | 141 | 63 | >5000 | >5000 | + |
| LC | 1.004 | 7.2 | 43.6 | 19 | 86 | 3850 | >5000 | + |
| LD | 1.003 | 6.5 | 24.7 | 37 | 27 | >5000 | >5000 | + |
| LF | 1.003 | 5.7 | 25.3 | 819 | 157 | >5000 | >5000 | + |
| LG | 1.024 | 5.4 | 351.8 | 601 | 1097 | >5000 | >5000 | + |
| LH | 1.014 | 5.5 | 147.7 | 49 | 13 | 4563 | 955 | + |
| LI | 1.010 | 6.6 | 148.3 | 159 | 150 | >5000 | >5000 | + |
| LJ | 1.008 | 7.9 | 96.4 | 27 | 31 | >5000 | 848 | + |
| LK | 1.004 | 7.2 | 22.7 | 234 | 32 | >5000 | 1839 | + |
| LM | 1.015 | 6.1 | 134.7 | 784 | 208 | >5000 | >5000 | + |
| LN | 1.015 | 6.5 | 212.7 | 233 | 89 | >5000 | 2618 | + |

ND: Indicates that the listed parent or metabolite compound was either not detected or detected below the established cut-off of 5 ng/mL indicating a negative result for that particular subject.

TABLE 16
Sample Validity and Urine Results Summary at 400 mg/day (all parent and
metabolite data reported in ng/mL).

| | | | | | | Quetiapine | |
|---|---|---|---|---|---|---|---|
| | Specific | | Creatinine | | 7-Hydroxy | Carboxylic | Quetiapine |
| | Gravity | pH | (mg/dL) | Quetiapine | Quetiapine | Acid | Sulfoxide |
| Average | 1.010 | 6.5 | 118.7 | 288 | 229 | 896 | 1442 |
| Standard | 0.005 | 0.8 | 80.1 | 380 | 273 | 1403 | 1540 |
| Deviation | | | | | | | |
| Maximum | 1.024 | 8.2 | 351.8 | 1616 | 1097 | >5000 | >5000 |
| Value | | | | | | | |
| Median | 1.010 | 6.4 | 96.4 | 124 | 148 | 412 | 874 |
| Value | | | | | | | |
| Minimum | 1.002 | 5.4 | 22.7 | 7 | 8 | 9 | 5 |
| Value | | | | | | | |

### Example 9

The urine of 9 patients who were prescribed 600 mg of Seroquel® (quetiapine) was tested for compliance (Table 17). 100 μL of each patient specimen was diluted with 50 μL of 1.6 μg/mL of quetiapine-D8 (internal standard) in methanol and 350 μL of 0.1% formic acid. Samples were subsequently vortexed and centrifuged prior to the injection of 5 μL on the QQQ.

The preponderance of quetiapine carboxylic acid and quetiapine sulfoxide in the urine as the major metabolic urine compounds is shown in Table 17. Assuming 9 opportunities to determine the patient to be either positive or negative for Seroquel® dosing, the use of quetiapine carboxylic acid and quetiapine sulfoxide resulted in ˜100% correct identification of those taking the prescribed medicine. The data in Table 18 demonstrate the “normal” nature of the sample validity criteria (i.e., pH, specific gravity, and creatinine). Without the quetiapine carboxylic acid and quetiapine sulfoxide metabolites, ˜78% were determined to be positive solely by the parent compound quetiapine and ˜89% were determined to be positive when using the parent compound quetiapine in conjunction with 7-hydroxy quetiapine. Using all 4 analytes resulted in 100% correct identification of those taking the prescribed medicine. Thus, use of quetiapine carboxylic acid and quetiapine sulfoxide as a urine biomarker at this dose does add value to compliance monitoring for Seroquel®.

TABLE 17
Test Results from Patients Prescribed 600 mg/day Seroquel ® (all parent
and metabolite data reported in ng/mL).

| | | | | | | Quetiapine | | |
|---|---|---|---|---|---|---|---|---|
| | Specific | | Creatinine | | 7-Hydroxy | Carboxylic | Quetiapine | All 4 |
| Subject | Gravity | pH | (mg/dL) | Quetiapine | Quetiapine | Acid | Sulfoxide | mets |
| LO | 1.007 | 7.1 | 104.1 | 13 | 59 | >5000 | 815 | + |
| LP | 1.006 | 7.5 | 101.4 | 288 | 1919 | >5000 | >5000 | + |
| LQ | 1.019 | 5.3 | 384.4 | ND | ND | 6 | ND | + |
| LR | 1.009 | 6.5 | 203.6 | 77 | 10 | >5000 | 1252 | + |
| LS | 1.010 | 7.1 | 215.2 | 4999 | 1104 | >5000 | >5000 | + |
| LT | 1.005 | 7.1 | 23.0 | 103 | 295 | >5000 | >5000 | + |
| LU | 1.006 | 7.0 | 33.9 | ND | 6 | 377 | 73 | + |
| LV | 1.010 | 6.1 | 129.5 | 1026 | 214 | 1598 | >5000 | + |
| LW | 1.007 | 6.2 | 86.7 | 18 | 19 | 4050 | 592 | + |

ND: Indicates that the listed parent or metabolite compound was either not detected or detected below the established cut-off of 5 ng/mL indicating a negative result for that particular subject.

TABLE 18
Sample Validity and Urine Results Summary at 600 mg/day (all parent and
metabolite data reported in ng/mL).

| | | | | | | Quetiapine | |
|---|---|---|---|---|---|---|---|
| | Specific | | Creatinine | | 7-Hydroxy | Carboxylic | Quetiapine |
| | Gravity | pH | (mg/dL) | Quetiapine | Quetiapine | Acid | Sulfoxide |
| Average | 1.009 | 6.7 | 142.4 | 932 | 453 | 1508 | 683 |
| Standard | 0.004 | 0.6 | 105.4 | 1693 | 651 | 1582 | 425 |
| Deviation | | | | | | | |
| Maximum | 1.019 | 7.5 | 384.4 | 4999 | 1919 | >5000 | >5000 |
| Value | | | | | | | |
| Median | 1.007 | 7.0 | 104.1 | 103 | 136 | 988 | 704 |
| Value | | | | | | | |
| Minimum | 1.005 | 5.3 | 23.0 | 13 | 6 | 6 | 73 |
| Value | | | | | | | |

### Example 10

The urine of 4 patients who were prescribed 800 mg of Seroquel® (quetiapine) was tested for compliance (Table 19). 100 μL of each patient specimen was diluted with 50 μL of 1.6 μg/mL of quetiapine-D8 (internal standard) in methanol and 350 μL of 0.1% formic acid. Samples were subsequently vortexed and centrifuged prior to the injection of 5 μL on the QQQ.

The preponderance of quetiapine carboxylic acid and quetiapine sulfoxide in the urine as the major metabolic urine compounds is shown in Table 19. Assuming 4 opportunities to determine the patient to be either positive or negative for Seroquel® dosing, the use of quetiapine carboxylic acid and quetiapine sulfoxide resulted in ˜100% correct identification of those taking the prescribed medicine. The data in Table 20 demonstrate the “normal” nature of the sample validity criteria (i.e., pH, specific gravity, and creatinine). Without the quetiapine carboxylic acid and quetiapine sulfoxide metabolites, ˜75% were determined to be positive solely by the parent compound quetiapine and ˜100% were determined to be positive when using the parent compound quetiapine in conjunction with 7-hydroxy quetiapine. Using all 4 analytes resulted in 100% correct identification of those taking the prescribed medicine.

TABLE 19
Test Results from Patients Prescribed 800 mg/day Seroquel ® (all parent
and metabolite data reported in ng/mL).

| | | | | | | Quetiapine | | |
|---|---|---|---|---|---|---|---|---|
| | Specific | | Creatinine | | 7-Hydroxy | Carboxylic | Quetiapine | All 4 |
| Subject | Gravity | pH | (mg/dL) | Quetiapine | Quetiapine | Acid | Sulfoxide | mets |
| LX | 1.003 | 7.3 | 57.3 | 137 | 512 | >5000 | >5000 | + |
| LY | 1.008 | 6.5 | 81.8 | 27 | 262 | >5000 | 1906 | + |
| LZ | 1.010 | 6.0 | 233.1 | ND | 48 | 52 | 7 | + |
| MA | 1.017 | 5.7 | 165.6 | 524 | 460 | >5000 | >5000 | + |

ND: Indicates that the listed parent or metabolite compound was either not detected or detected below the established cut-off of 5 ng/mL indicating a negative result for that particular subject.

TABLE 20
Sample Validity and Urine Results Summary at 800 mg/day (all parent and
metabolite data reported in ng/mL).

| | | | | | | Quetiapine | |
|---|---|---|---|---|---|---|---|
| | Specific | | Creatinine | | 7-Hydroxy | Carboxylic | Quetiapine |
| | Gravity | pH | (mg/dL) | Quetiapine | Quetiapine | Acid | Sulfoxide |
| Average | 1.010 | 6.4 | 134.5 | 229 | 320 | 52 | 957 |
| Standard | 0.005 | 0.6 | 69.7 | 213 | 183 | 0 | 950 |
| Deviation | | | | | | | |
| Maximum | 1.017 | 7.3 | 233.1 | 524 | 512 | >5000 | >5000 |
| Value | | | | | | | |
| Median | 1.009 | 6.3 | 123.7 | 137 | 361 | 52 | 957 |
| Value | | | | | | | |
| Minimum | 1.003 | 5.7 | 57.3 | 27 | 48 | 52 | 7 |
| Value | | | | | | | |

### Example 11

The urine of 29 patients who were prescribed various doses of Seroquel® (quetiapine) was tested for compliance after enzymatic hydrolysis (Table 21). 100 μL of each patient specimen was diluted with 400 μL of a “master mix” that included 1.6 μg/mL of quetiapine-D8 (internal standard), 0.06 M phosphate buffer, and at least 1450 U of a recombinant β-glucuronidase enzyme. Samples were subsequently vortexed, incubated for 1 hr at ˜65° C., and then centrifuged prior to the injection of 5 μL on the QQQ.

The preponderance of quetiapine carboxylic acid and quetiapine sulfoxide post-hydrolysis in the urine as the major metabolic urine compounds is shown in Table 21. Assuming 29 opportunities to determine the patient to be either positive or negative for Seroquel® dosing, the use of quetiapine carboxylic acid and quetiapine sulfoxide resulted in ˜100% correct identification of those taking the prescribed medicine with or without enzymatic hydrolysis. The data in Table 22 demonstrate the “normal” nature of the sample validity criteria (i.e., pH, specific gravity, and creatinine). Without the quetiapine carboxylic acid and quetiapine sulfoxide metabolites, ˜69% were determined to be positive solely by the parent compound quetiapine pre-hydrolysis and ˜97% were determined to be positive solely by the parent compound quetiapine post-hydrolysis. Using quetiapine in conjunction with 7-hydroxy quetiapine 79% were determined to be positive pre-hydrolysis and ˜97% were determined to be positive post-hydrolysis.

TABLE 21
Test Results from Patients Prescribed Seroquel ® Post-Hydrolysis (all
parent and metabolite data reported in ng/mL).

| | Pre-Hydrolysis Results | | | | Post-Hydrolysis Results | | | |
|---|---|---|---|---|---|---|---|---|
| | | | Quetiapine | | | | Quetiapine | |
| | | 7-Hydroxy | Carboxylic | Quetiapine | | 7-Hydroxy | Carboxylic | Quetiapine |
| Subject | Quetiapine | Quetiapine | Acid | Sulfoxide | Quetiapine | Quetiapine | Acid | Sulfoxide |
| B | ND | ND | 9 | ND | 67 | 7 | 187 | 25 |
| T | ND | ND | 6 | ND | 86 | 7 | 176 | 24 |
| W | ND | ND | 5 | ND | ND | ND | 12 | ND |
| AJ | ND | ND | 5 | ND | 29 | ND | 156 | 24 |
| AQ | 1118 | 694 | >5000 | 740 | >5000 | 1853 | >5000 | 863 |
| BY | 17 | 51 | >5000 | 2143 | 4059 | 298 | >5000 | 2457 |
| CG | ND | 357 | >5000 | >5000 | 1494 | 444 | >5000 | >5000 |
| CH | ND | 258 | >5000 | >5000 | 2579 | 530 | >5000 | >5000 |
| ED | ND | ND | 10 | ND | 14 | ND | 76 | 13 |
| FV | 208 | 262 | >5000 | >5000 | >5000 | 623 | >5000 | >5000 |
| FW | 315 | 29 | >5000 | >5000 | >5000 | 616 | >5000 | >5000 |
| FX | 97 | 5 | >5000 | 2971 | 3019 | 78 | >5000 | 2505 |
| GF | 187 | 262 | >5000 | >5000 | 2928 | 556 | >5000 | >5000 |
| GG | 222 | 116 | >5000 | >5000 | 1271 | 309 | >5000 | >5000 |
| HQ | 269 | 133 | >5000 | >5000 | >5000 | 516 | >5000 | >5000 |
| HR | 4574 | >5000 | >5000 | >5000 | >5000 | 3588 | >5000 | >5000 |
| HS | 755 | >5000 | >5000 | >5000 | >5000 | 3588 | >5000 | >5000 |
| HY | 917 | 1961 | >5000 | >5000 | >5000 | 3751 | >5000 | >5000 |
| IF | ND | 200 | >5000 | >5000 | 2369 | 311 | >5000 | 3866 |
| IH | 361 | 63 | >5000 | >5000 | >5000 | 2545 | >5000 | >5000 |
| IK | 873 | 2034 | >5000 | >5000 | >5000 | >5000 | >5000 | >5000 |
| JY | 77 | 324 | >5000 | 4484 | >5000 | >5000 | >5000 | >5000 |
| KG | 304 | 580 | >5000 | >5000 | >5000 | 1185 | >5000 | >5000 |
| KH | 1137 | 958 | >5000 | >5000 | >5000 | 3152 | >5000 | >5000 |
| KV | 120 | 39 | >5000 | >5000 | 3028 | 3941 | >5000 | >5000 |
| LQ | ND | ND | 6 | ND | 15 | ND | 56 | 8 |
| LR | 77 | 10 | >5000 | 1252 | 2147 | 509 | >5000 | 1541 |
| LS | 4999 | 1104 | >5000 | >5000 | >5000 | 2738 | >5000 | >5000 |
| LX | 137 | 512 | >5000 | >5000 | 4911 | 976 | >5000 | >5000 |
| Average | 838 | 474 | 7 | 2318 | 1868 | 1397 | 111 | 1133 |
| Standard Deviation | 1362.9 | 577.3 | 2.0 | 1324.8 | 1546.1 | 1360.2 | 65.9 | 1324.8 |
| Maximum Value | 4999 | >5000 | >5000 | >5000 | >5000 | >5000 | >5000 | >5000 |
| Median Value | 287 | 262 | 6 | 2143 | 2147 | 616 | 116 | 444 |
| Minimum Value | 17 | 5 | 5 | 740 | 14 | 7 | 12 | 8 |

ND: Indicates that the listed parent or metabolite compound was either not detected or detected below the established cut-off of 5 ng/mL indicating a negative result for that particular subject.

TABLE 22
Sample Validity and Urine Results Summary for Post-Hydrolysis.

| | Specific Gravity | pH | Creatinine (mg/dL) |
|---|---|---|---|
| Average | 1.013 | 6.3 | 165.2 |
| Standard Deviation | 0.005 | 0.8 | 86.9 |
| Maximum Value | 1.022 | 8.0 | 384.4 |
| Median Value | 1.013 | 6.2 | 139.4 |
| Minimum Value | 1.003 | 4.9 | 31.6 |

From the foregoing, it will be appreciated that specific embodiments of the invention have been described herein for purposes of illustration, but that various modifications may be made without deviating from the scope of the invention. Accordingly, the invention is not limited except as by the appended claims.

1. A method for detecting quetiapine in a fluid sample obtained from a subject that has been prescribed quetiapine comprising:
detecting a quetiapine metabolite in the fluid sample via QQQ mass spectrometry or Q-ToF mass spectrometry; and
detecting quetiapine in the fluid sample when at least 5 ng/mL of the quetiapine metabolite is detected in the sample, wherein the quetiapine metabolite is one or more of quetiapine, quetiapine sulfoxide, N-desalkylquetiapine, 7-hyroxyquetiapine, 7-hydroxyquetiapine glucuronide, quetiapine carboxylic acid, quetiapine glucuronide, quetiapine sulfoxide glucuronide, and quetiapine carboxylic acid glucuronide.

2. (canceled)

3. (canceled)

4. The method of claim 1, wherein about 5 ng/mL, about 20 ng/mL, or about 50 ng/mL is detected in the fluid sample.

5. The method of claim 1, wherein the fluid sample is a urine sample.

6. (canceled)

7. The method of claim 1, wherein the quetiapine metabolite is two or more of: quetiapine, quetiapine sulfoxide, N-desalkylquetiapine, 7-hyroxyquetiapine, 7-hydroxyquetiapine glucuronide, quetiapine carboxylic acid, quetiapine glucuronide, quetiapine sulfoxide glucuronide, and quetiapine carboxylic acid glucuronide.

8. The method of claim 1, wherein the quetiapine metabolite is three or more of: quetiapine, quetiapine sulfoxide, N-desalkylquetiapine, 7-hyroxyquetiapine, 7-hydroxyquetiapine glucuronide, quetiapine carboxylic acid, quetiapine glucuronide, quetiapine sulfoxide glucuronide, and quetiapine carboxylic acid glucuronide.

9. The method of claim 1, wherein the quetiapine metabolite is four or more of: quetiapine, quetiapine sulfoxide, N-desalkylquetiapine, 7-hyroxyquetiapine, 7-hydroxyquetiapine glucuronide, quetiapine carboxylic acid, quetiapine glucuronide, quetiapine sulfoxide glucuronide, and quetiapine carboxylic acid glucuronide.

10. (canceled)

11. (canceled)

12. The method of claim 1 further comprising contacting the fluid sample with a hydrolyzing enzyme before detecting the presence of the quetiapine metabolite in the fluid sample.

13. The method of claim 12, wherein the hydrolyzing enzyme is a glucuronidase enzyme.

14. The method of claim 13, wherein the glucuronidase enzyme is a recombinant β-glucuronidase enzyme.

15. (canceled)

16. (canceled)

17. (canceled)

18. A method for detecting quetiapine in a fluid sample obtained from a subject that has been prescribed quetiapine comprising:
detecting a quetiapine metabolite in the fluid sample via QQQ mass spectrometry or Q-ToF mass spectrometry, wherein the fluid sample has been hydrolyzed; and
detecting quetiapine in the fluid sample when at least 5 ng/mL of the quetiapine metabolite is detected in the sample, wherein the quetiapine metabolite is one or more of quetiapine, quetiapine sulfoxide, 7-hyroxyquetiapine, and quetiapine carboxylic acid.

19. The method of claim 18, wherein the fluid sample is hydrolyzed by contacting the fluid sample with a composition comprising a glucuronidase enzyme.

20. The method of claim 19, wherein the glucuronidase enzyme is a recombinant β-glucuronidase enzyme.

21. (canceled)

22. The method of claim 18, wherein the hydrolyzed fluid sample does not contain any one of quetiapine, quetiapine sulfoxide, 7-hyroxyquetiapine, and quetiapine carboxylic acid in an amount that is greater than 5 ng/mL.

23. (canceled)

24. (canceled)

25. (canceled)

26. The method of claim 1, wherein the subject has been prescribed a quetiapine dose selected from the group consisting of 800 mg/day, 600 mg/day, 400 mg/day, 300 mg/day, 200 mg/day, 150 mg/day, 100 mg/day, 50 mg/day, and 25 mg/day.

27. The method of claim 18, wherein the subject has been prescribed a quetiapine dose selected from the group consisting of 800 mg/day, 600 mg/day, 400 mg/day, 300 mg/day, 200 mg/day, 150 mg/day, 100 mg/day, 50 mg/day, and 25 mg/day.
